(19) United States
(12) Patent Application Publication
(10) Pub. No.: US 2025/0367385 A1
(43) Pub. Date: Dec. 4, 2025

(54) MEDICAL INJECTIONS AND RELATED DEVICES AND METHODS

(71) Applicant: Gilead Sciences, Inc., Foster City, CA (US)

(72) Inventors: Stephan Edward Augustyn, Cambridge (GB); Berk Besen, Cambridge (GB); Stuart Curtis, Cambridge (GB); Rosemary Victoria Earl, Cambridge (GB); Walter Goodwin, San Carlos, CA (US); Matthew Robert Hiskey, Cambridge (GB); William James Kirby, Cambridge (GB); Finlay Knops-McKim, Cambridge (GB); Daniel Milton Meruz, Cambridge (GB); James Paul Oberhauser, Saratoga, CA (US); Alasdair Young, Foster City, CA (US)

(21) Appl. No.: 19/206,132

(22) Filed: May 13, 2025

Related U.S. Application Data
(60) Provisional application No. 63/647,292, filed on May 14, 2024.

(51) Int. Cl. A61M 5/315; A61K 9/00; A61K 31/4439; A61K 47/10; A61M 5/20

(52) U.S. Cl. CPC A61M 5/3157; A61K 9/0019; A61K 31/4439; A61K 47/10; A61M 5/2046; A61M 2005/31588; A61M 2205/3317; A61M 2205/3368; A61M 2205/581

(57) ABSTRACT

Systems, devices, and methods include an autoinjector including a housing; a needle arranged at a distal end of the housing; a container disposed within the housing and configured to contain medicament; and a plunger slidably disposed within the container. A plunger rod is configured to push the plunger through the container to dispense the medicament through the needle when the container contains medicament. The plunger rod includes one or more conductive regions. A mechanism is configured so that a dispensing movement of the plunger rod causes the mechanism to generate a first electrical signal from at least one of the one or more conductive regions. A first sensor is configured to detect the first electrical signal generated by the mechanism.

### CROSS REFERENCE TO RELATED APPLICATIONS

This application claims priority to U.S. Provisional Application No. 63/647,292, filed May 14, 2024, the entire content of which application is hereby incorporated by reference in its entirety.

### TECHNICAL FIELD

This disclosure relates to medical injections and related devices and methods.

### BACKGROUND

An injection typically refers to the act of administering a liquid (e.g., a drug) into a patient's body tissue. Injecting medicament into a patient can allow the medicament to be absorbed relatively rapidly.

### SUMMARY

The present systems, devices, and methods can sense a dispensing movement of a plunger rod within an autoinjector. For example, a pattern of conductive regions (or conductive elements) on the plunger rod of the autoinjector can produce signals that can be sensed by a sensing assembly to track the dispensing movement of the plunger rod. The signals can include voltage or other electrical measurements that indicate that a conductive region has reached a certain location within the autoinjector or that a certain number of conductive regions has progressed through the autoinjector. The devices can include a sensing assembly that can determine that the plunger rod has completed the dispensing movement after receiving a predetermined number of signals. The sensing assembly can also track the dispensing movement of the plunger rod. Tracking the dispensing movement of the plunger rod can be advantageous because the dispensing movement of the plunger rod corresponds to the amount of medicament delivered to the subject (e.g., a patient). If the plunger rod does not complete the dispensing movement, then the full dose of medicament is not injected into the patient. The sensing assembly can be used to determine that the full dose is injected into the patient by tracking the dispensing movement. Tracking movement of the plunger rod throughout the dispensing movement can be advantageous for determining how much of a medicament has been delivered and whether the amount of medicament injected exceeds a minimum dose volume threshold for medicament efficacy.

The present systems, devices, and methods can also sense whether a needle of the autoinjector is inserted into a patient to a sufficient depth and/or whether the insertion depth is maintained during delivery of the medicament. For example, an electrical contact between a needle guard and a particular location in the autoinjector can determine whether the needle has been inserted to a sufficient depth. Determining that the needle is sufficiently inserted into the patient can be advantageous because if the needle is not sufficiently inserted into the patient, the patient may not receive the medicament properly.

The present disclosure relates to a composition comprising lenacapavir or a pharmaceutically accepted salt thereof for use in the prevention or treatment of HIV, where the lenacapavir is administered by an autoinjector according to this disclosure. The present disclosure also relates to a use of lenacapavir or a pharmaceutically accepted salt thereof for the manufacture of a medicament for the prevention or treatment of HIV, where the lenacapavir is administered by an autoinjector according to the disclosure.

In a first aspect of the invention, the present disclosure encompasses an autoinjector including a housing; a needle arranged at a distal end of the housing; a container disposed within the housing and configured to contain medicament; a plunger slidably disposed within the container; a plunger rod configured to push the plunger through the container to dispense the medicament through the needle when the container contains medicament, where the plunger rod includes one or more conductive regions; a first conductive contact configured to contact a portion of the plunger rod, where a dispensing movement of the plunger rod generates a first electrical signal upon contacting the first conductive contact and at least one of the one or more conductive regions of the plunger rod; and a first sensor configured to detect the first electrical signal as the plunger rod moves.

According to the first aspect of the invention, the first electrical signal may be indicative of dose progression.

According to the first aspect of the invention, the container may contain the medicament. Additionally or alternatively, the medicament may comprise lenacapavir or a pharmaceutically accepted salt thereof.

According to the first aspect of the invention, the autoinjector may further comprise a gas canister assembly configured to release pressurized gas which, when released, provides a force acting on the plunger rod to push the plunger through the container.

According to the first aspect of the invention, the first conductive contact may include a lock ring contact configured to be attached to a lock ring. The lock ring contact and/or the lock ring may include an opening configured to provide a pathway for the dispensing movement of the plunger rod. The lock ring contact may include one or more deflectable contacts disposed around the opening and configured to contact at least one of the one or more conductive regions during the dispensing movement of the plunger rod.

According to the first aspect of the invention, the plunger rod may be configured to generate a plurality of electrical signals during the dispensing movement of the plunger rod. The plunger rod may include a plurality of conductive regions. Additionally or alternatively, the first electrical signal may be configured to indicate completion of the dispensing movement of the plunger rod. A first conductive region of the plurality of conductive regions may be configured to indicate dose start of the medicament, and/or a last conductive region of the plurality of conductive regions may be configured to indicate dose end of the medicament. Additionally or alternatively, each conductive region of the plurality of conductive regions is configured to generate a corresponding electrical signal during the dispensing movement of the plunger rod.

According to the first aspect of the invention, the first conductive contact may be configured to generate a plurality of electrical signals during the dispensing movement of the plunger rod. The first conductive contact may include an opening and one or more deflectable contacts disposed around the opening. The one or more deflectable contacts may be configured to contact each conductive region during the dispensing movement of the plunger rod.

According to the first aspect of the invention, the autoinjector may include an audible clicker configured to produce one or more audible clicks during the dispensing movement of the plunger rod. The audible clicker may comprise a ring surrounding the plunger rod, and wherein the ring comprises a deflectable protrusion configured to contact the plunger rod and produce the one or more audible clicks. The plunger rod may include a ridged surface configured to contact the deflectable protrusion of the audible clicker. Each ridge of the ridged surface may be configured to cause the deflectable protrusion to deflect during the dispensing movement of the plunger rod.

According to the first aspect of the invention, the first sensor may be disposed between a proximal end and a distal end of the housing. Additionally or alternatively, the first sensor may include a voltage sensor or a resistance sensor. Additionally or alternatively, the first sensor may be disposed on a surface of a printed circuit board. Additionally or alternatively, the first sensor may be further configured to detect a second electrical signal generated by applying an insertion force to insert the needle into a user.

According to the first aspect of the invention, a second sensor may be disposed between a proximal end and a distal end of the housing, and wherein the second sensor may be configured to detect a second electrical signal generated by applying an insertion force to insert the needle into a user. The second sensor may include a voltage sensor or a resistance sensor.

According to the first aspect of the invention, a temperature sensor may be disposed between a proximal end and a distal end of the housing, and wherein the temperature sensor may be configured to detect an ambient temperature in proximity to the container. The temperature sensor may include a thermistor.

In a second aspect of the invention, the present disclosure encompasses an autoinjector including a housing; a needle arranged at a distal end of the housing; a container disposed within the housing and configured to contain medicament; a plunger slidably disposed within the container; a plunger rod configured to push the plunger through the container to dispense the medicament through the needle when the container contains medicament, where the plunger rod includes one or more conductive regions; a mechanism configured so that a dispensing movement of the plunger rod causes the mechanism to generate a first electrical signal from at least one of the one or more conductive regions; and a first sensor configured to detect the first electrical signal generated by the mechanism.

In a third aspect of the invention, the present disclosure encompasses an autoinjector including a housing; a needle arranged at a distal end of the housing; a needle guard configured to expose the needle when an insertion force is applied to insert the needle into a user, where the needle guard includes a needle guard contact; a container disposed within the housing and configured to contain medicament; a plunger slidably disposed within the container; a plunger rod configured to push the plunger through the container to dispense the medicament through the needle when the container contains medicament and when the needle is exposed, where the plunger rod includes one or more conductive regions; a lock ring contact configured to contact a portion of the plunger rod, where a dispensing movement of the plunger rod generates a first electrical signal upon contacting the lock ring contact and at least one of the one or more conductive regions of the plunger rod, and where a compression movement of the needle guard generates a second electrical signal upon contacting the lock ring contact and the needle guard contact; and a first sensor configured to detect the first electrical signal as the plunger rod moves and/or said second electrical signal as the needle guard moves.

In a fourth aspect of the invention, the present disclosure encompasses a system including an autoinjector (e.g., any described herein, such as those of the first, second or third aspects of the invention); and a processor configured to process data generated by the sensor to track the dispensing movement of the plunger rod.

In a fifth aspect of the invention, the present disclosure encompasses a method of detecting a dispensing movement of a plunger rod within an autoinjector including detecting, using a first sensor, one or more electrical signals of a first conductive contact as the plunger rod moves during the dispensing movement, where the plunger rod includes one or more conductive regions, and where the dispensing movement of the plunger rod provides electrical communication between one or more conductive regions and the first conductive contact causing the first conductive contact to generate the one or more electrical signals.

In a sixth aspect of the invention, the present disclosure encompasses a method of detecting a dispensing movement of a plunger rod within an autoinjector including detecting an electrical signal generated due to movement of the plunger rod during the dispensing movement.

According to the fifth or sixth aspect of the invention, the plunger rod may be configured to generate a plurality of electrical signals during the dispensing movement of the plunger rod. The plunger rod may include a plurality of conductive regions. A first conductive region of the plurality of conductive regions may be configured to indicate dose start of the medicament, and/or a last conductive region of the plurality of conductive regions may be configured to indicate dose end of the medicament. Additionally or alternatively, each conductive region of the plurality of conductive regions may be configured to generate a corresponding electrical signal during the dispensing movement of the plunger rod.

According to the fifth or sixth aspects of the invention, a lock ring contact may be configured to generate a plurality of electrical signals during the dispensing movement of the plunger rod. The lock ring contact may comprise an opening and one or more deflectable contacts disposed around said opening. The one or more deflectable contacts may be configured to contact each conductive region during the dispensing movement of the plunger rod.

According to the fifth or sixth aspects of the invention, the autoinjector may further include an audible clicker configured to produce one or more audible clicks during the dispensing movement of the plunger rod. The audible clicker may include a ring surrounding the plunger rod, and where the ring includes a deflectable protrusion configured to contact the plunger rod and produce the one or more audible clicks. The plunger rod may include a ridged surface configured to contact the deflectable protrusion of the audible clicker. Each ridge of the ridged surface may be configured to cause the deflectable protrusion to deflect during the dispensing movement of the plunger rod.

According to the fifth aspect of the invention, the first sensor is disposed between a proximal end and a distal end of the housing. Additionally or alternatively, the first sensor may include a voltage sensor. Additionally or alternatively, the first sensor may be disposed on a surface of a printed circuit board. Additionally or alternatively, the first sensor may be further configured to detect a second electrical signal generated by applying an insertion force to insert the needle into a user.

According to the fifth or sixth aspects of the invention, a second sensor may be disposed between a proximal end and a distal end of the housing, and wherein the second sensor may be configured to detect a second electrical signal generated by applying an insertion force to insert the needle into a user. The second sensor may include a voltage sensor or a resistance sensor.

According to the fifth or sixth aspects of the invention, a temperature sensor may be disposed between a proximal end and a distal end of the housing, and where the temperature sensor may be configured to detect an ambient temperature in proximity to the container. The temperature sensor may include a thermistor.

The fifth or sixth aspects of the invention may include sending, via a wireless transfer protocol module, signals from a first sensor, if present, and a second sensor, if present, to a mobile device. Additionally, the fifth or sixth aspects of the invention may include displaying information about the dispensing movement on the mobile device.

The fifth or sixth aspects of the invention may include measuring a temperature of medicament within the autoinjector or an ambient temperature within the autoinjector using a temperature sensor. The fifth or sixth aspects of the invention may further include sending, via a wireless transfer protocol module, signals from the temperature sensor to a mobile device. Additionally or alternatively, the fifth or sixth aspects of the invention may include displaying the temperature of the medicament or the ambient temperature on the mobile device; indicating that the temperature of the medicament or the ambient temperature is above a threshold temperature for use of the autoinjector; or both.

According to a seventh aspect of the invention, also provided is a composition comprising lenacapavir or a pharmaceutically accepted salt thereof for use in the prevention or treatment of HIV, where the composition is administered via an autoinjector (e.g., any described herein, such as those of the first, second or third aspects of the invention). The administration may be subcutaneous or intramuscular.

According to an eighth aspect of the invention, also provided is the use of lenacapavir or a pharmaceutically accepted salt thereof for the manufacture of a medicament for the prevention or treatment of HIV, wherein the prevention or treatment comprises administering the medicament subcutaneously via an autoinjector (e.g., any described herein, such as those of the first, second or third aspects of the invention). The administration may be subcutaneous or intramuscular.

The details of one or more embodiments of the subject matter described herein are set forth in the accompanying drawings and the description below. Other features, objects, and advantages of the subject matter will be apparent from the description and drawings, and from the claims.

### DESCRIPTION OF DRAWINGS

FIG. 1 illustrates an example of an autoinjector.

FIG. 2 is a cut-away view of an example autoinjector.

FIG. 3A illustrates an example needle guard of the autoinjector of FIG. 2.

FIG. 3B illustrates an example lock ring of the autoinjector of FIG. 2.

FIG. 3C illustrates an example syringe carrier of the autoinjector of FIG. 2.

FIG. 3D illustrates an example transfer sleeve of the autoinjector of FIG. 2.

FIG. 3E illustrates an example plunger rod of the autoinjector of FIG. 2.

FIG. 3F is an exploded view of the autoinjector of FIG. 2.

FIG. 4A illustrates a portion of the autoinjector of FIG. 2.

FIG. 4B illustrates a portion of an example autoinjector with a different printed circuit board (PCB) location than the autoinjector of FIG. 2.

FIG. 5 is a cut-away view of the autoinjector of FIG. 4B.

FIG. 6 is an exploded view of the autoinjector of FIG. 4B.

FIG. 7 is a cross section view of an example autoinjector.

FIG. 8 illustrates an example autoinjector during operation of the autoinjector.

FIG. 9 is an example workflow for laser direct structuring.

FIG. 10 illustrates another example autoinjector.

FIGS. 11A-E illustrate a plunger and a lock ring of the autoinjector of FIG. 10.

FIG. 12 illustrates a process of dispensing a dose of medicament using the autoinjector of FIG. 10.

FIG. 13A is an exploded view of the autoinjector of FIG. 10.

FIG. 13B is a detail view of the PCB and needle guard of the autoinjector of FIG. 10.

FIG. 13C is an example workflow for assembling the autoinjector of FIG. 10.

FIG. 14 is a flow chart for an example method of dispensing a dose using an autoinjector.

FIG. 15 is an example plot of detected voltage indicating dose progression for an example autoinjector.

FIG. 16 is an example plot of temperature readings of a medicament using an example autoinjector.

Like reference numbers and designations in the various drawings indicate like elements.

### DETAILED DESCRIPTION

The present systems, devices, and methods can sense a dispensing movement of a plunger rod within an autoinjector. For example, a pattern of conductive regions on the plunger rod of the autoinjector can produce signals that can be sensed by a sensing assembly to track the dispensing movement of the plunger rod. The signals can include voltage measurements or other electrical measurements (e.g., resistance measurements and the like) that indicate that a conductive region has reached a certain location within the autoinjector or that a certain number of conductive regions has progressed through the autoinjector. The devices can include a sensing assembly that can determine that the plunger rod has completed the dispensing movement after receiving a predetermined number of signals. The sensing assembly can also track the dispensing movement of the plunger rod. Tracking the dispensing movement of the plunger rod can be advantageous because the dispensing movement of the plunger rod corresponds to the amount of medicament delivered to the patient. If the plunger rod does not complete the dispensing movement, then the full dose of medicament is not injected into the patient. The sensing assembly can be used to determine that the full dose is injected into the patient by tracking the dispensing movement. Tracking movement of the plunger rod throughout the dispensing movement can be advantageous for determining how much of a medicament has been delivered and whether the amount of medicament injected exceeds a minimum dose volume threshold for medicament efficacy.

The present systems, devices, and methods can also sense whether a needle of the autoinjector is inserted into a patient to a sufficient depth. For example, an electrical contact between a needle guard and a particular location in the autoinjector can determine whether the needle has been inserted to a sufficient depth. Determining that the needle is sufficiently inserted into the patient can be advantageous because if the needle is not sufficiently inserted into the patient, the patient may not receive the medicament properly.

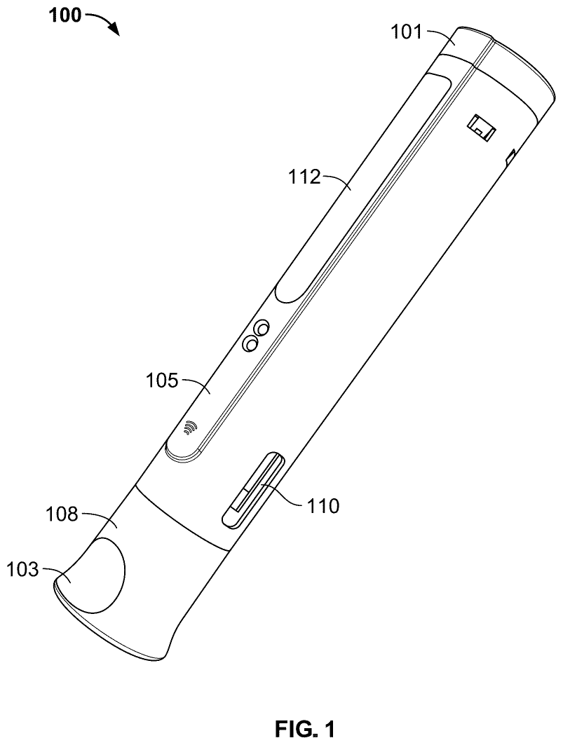

FIG. 1 illustrates an autoinjector 100 that can sense a dispensing movement of a plunger rod within the autoinjector 100 and sense whether a needle of the autoinjector 100 is inserted into a patient to a sufficient depth. Alternatively, or in addition, the autoinjector 100 can sense whether an insertion depth of a needle is maintained during dispensing or delivery. The autoinjector 100 includes a housing 105, a proximal end 101 of the housing 105, and a front cap 108 attached to a distal end 103 of the housing 105. The front cap 108 covers a needle assembly configured to be inserted into the user during injection. The user removes the front cap 108 prior to use of the autoinjector 100. A sensing assembly can be located somewhere between the proximal end 101 and the distal end 103 of the housing 105. The sensing assembly can sense a dispensing movement of a plunger rod within the autoinjector 100, e.g., to determine that the plunger rod has completed the dispensing movement, as discussed below. The housing 105 can include flanges configured to accommodate the user's fingers.

The housing 105 can include an optional label 112 that provides information about the autoinjector 100. For example, the label 112 can include medicament information, such as the type of medicament, the size of the dose, and the delivery time of the dose. Optionally, the housing 105 does not include a label 112.

The housing 105 also includes an optional window 110, through which a user can see medicament contained within the autoinjector 100, e.g., within a container of the autoinjector 100 (see discussion below). The window 110 may help a user determine whether the autoinjector 100 has been used. Before use of the autoinjector 100, the user can see through the window 110 to determine whether there is medicament within the autoinjector 100, for example, to determine that the autoinjector has not been used. During use of the autoinjector 100, the user may look through the window 110 to determine whether the volume of medicament in the autoinjector 100 is decreasing. After use of the autoinjector 100, the user may look through the window 110 to determine that there is no medicament in the autoinjector 100, for example, to determine that the autoinjector 100 has been used.

Different injection sites, patient age and patient body mass may affect the recommended needle length, and higher viscosity drugs will require a larger diameter needle to prevent the injection force becoming too high for the device. Advantageously, selecting needle gauge based on viscosity of drug being administered can ensure that the full dose of drug is administered without undue strain. Typically, injection force is less than 40 Newtons through needle gauge selection. Preferably, injection force is less than 20 Newtons through needle gauge selection.

Needle gauges disclosed herein are provided in Birmingham Wire Gauge (also known as: Birmingham Gauge or Stubs Iron Wire Gauge), abbreviated as “gauge” or G. In accordance with ISO standard ISO 9626:2016, needle wall thickness designations include Regular Wall, Thin Wall, Extra Thin Wall, and Ultra Thin Wall. Regular Wall thickness is abbreviated to RW. Thin Wall thickness is abbreviated to TW. Extra Thin Wall thickness is abbreviated to ETW. Ultra Thin Wall is abbreviated to UTW. Alternatively, needle wall thickness may be Special Thin Wall; Special Thin Wall thickness is abbreviated as STW. Viscosity is provided in centipoise (cP), where one centipoise is equivalent to one millipascal-second.

The autoinjector 100 may be used for subcutaneous injections, which are directed into fat tissue between the skin and the muscle of the patient. Subcutaneous injections typically involve shorter and narrower needles than intramuscular injections, which are directed into the muscle of a patient. Needles for subcutaneous injections are typically 34-27 gauge and 4-12 mm in insertion depth (needle extension) for subcutaneous injections into the abdomen. Insertion depth for a subcutaneous injection may be 4-8 mm. For subcutaneous injection, needle length may be 8-13 mm. For delivery of 2.25-3 mL dose of liquid (medicament) with a viscosity of up to 5 cP using a needle with a needle length 8-13 mm, needle gauge may be 29G RW or TW, or 27G RW. For delivery of 2.25-3 mL dose of liquid (medicament) with a viscosity of up to 30 cP using a needle with a needle length 8-13 mm, needle gauge may be 27G TW or 25G RW. For delivery of 2.25-3 mL dose of liquid (medicament) with a viscosity of up to 50 cP using a needle with a needle length 8-13 mm, needle gauge may be 25G TW. For delivery of 2.25-3 mL dose of liquid (medicament) with a viscosity of up to 160 cP using a needle with a needle length 8-13 mm, needle gauge may be 25G STW or 23G RW. For delivery of 2.25-3 mL dose of liquid (medicament) with a viscosity of up to 300 cP using a needle with a needle length 8-13 mm, needle gauge may be 22G ETW. For delivery of 2.25-3 mL dose of liquid (medicament) with a viscosity of up to 600 cP using a needle with a needle length 8-13 mm, needle gauge may be 18G ETW or 18G UTW. For delivery of 1 mL dose of liquid (medicament) with a viscosity of up to 10 cP using a needle with a needle length 8-13 mm, needle gauge may be 29G RW or TW, or 27G RW. For delivery of 1 mL dose of liquid (medicament) with a viscosity of up to 30 cP using a needle with a needle length 8-13 mm, needle gauge may be 27G TW or 25G RW. For delivery of 1 mL dose of liquid (medicament) with a viscosity of up to 80 cP using a needle with a needle length 8-13 mm, needle gauge may be 25G TW. For delivery of 1 mL dose of liquid (medicament) with a viscosity of up to 350 cP using a needle with a needle length 8-13 mm, needle gauge may be 25G STW or 23G RW. For delivery of 1 mL dose of liquid (medicament) with a viscosity of up to 600 cP using a needle with a needle length 8-13 mm, needle gauge may be 18-22G ETW.

The autoinjector 100 may be used for intramuscular injections. Needles for intramuscular injections on adults are typically 25-20 gauge and 15-25 mm in insertion depth (needle extension). Alternatively, insertion depth for an intramuscular injection may be 25-50 mm. For intramuscular injection, needle length may be 1-1.5 inches (25.4-38.1 mm). For delivery of 2.25-3 mL dose of liquid (medicament) with a viscosity of up to 1 cP using a needle with a needle length 25.4-38.1 mm, needle gauge may be 29G RW or TW, or 27G RW. For delivery of 2.25-3 mL dose of liquid (medicament) with a viscosity of up to 5 cP using a needle with a needle length 25.4-38.1 mm, needle gauge may be 27G TW or 25G RW. For delivery of 2.25-3 mL dose of liquid (medicament) with a viscosity of up to 10 cP using a needle with a needle length 25.4-38.1 mm, needle gauge may be 25G TW. For delivery of 2.25-3 mL dose of liquid (medicament) with a viscosity of up to 40 cP using a needle with a needle length 25.4-38.1 mm, needle gauge may be 25G STW or 23G RW. For delivery of 2.25-3 mL dose of liquid (medicament) with a viscosity of up to 200 cP using a needle with a needle length 25.4-38.1 mm, needle gauge may be 22G ETW. For delivery of 2.25-3 mL dose of liquid (medicament) with a viscosity of up to 600 cP using a needle with a needle length 25.4-38.1 mm, needle gauge may be 18G ETW. For delivery of 1 mL dose of liquid (medicament) with a viscosity of up to 5 cP using a needle with a needle length 25.4-38.1 mm, needle gauge may be 29G RW or TW, or 27G RW. For delivery of 1 mL dose of liquid (medicament) with a viscosity of up to 10 cP using a needle with a needle length 25.4-38.1 mm, needle gauge may be 27G TW or 25G RW. For delivery of 1 mL dose of liquid (medicament) with a viscosity of up to 30 cP using a needle with a needle length 25.4-38.1 mm, needle gauge may be 25G TW. For delivery of 1 mL dose of liquid (medicament) with a viscosity of up to 50 cP using a needle with a needle length 25.4-38.1 mm, needle gauge may be 25G STW or 23G RW. For delivery of 1 mL dose of liquid (medicament) with a viscosity of greater than 50 cP using a needle with a needle length 25.4-38.1 mm, needle gauge may be 18-22G ETW.

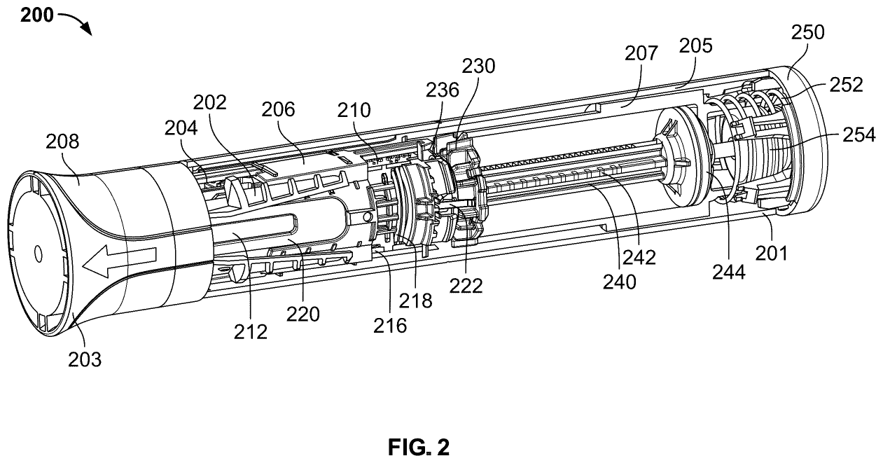

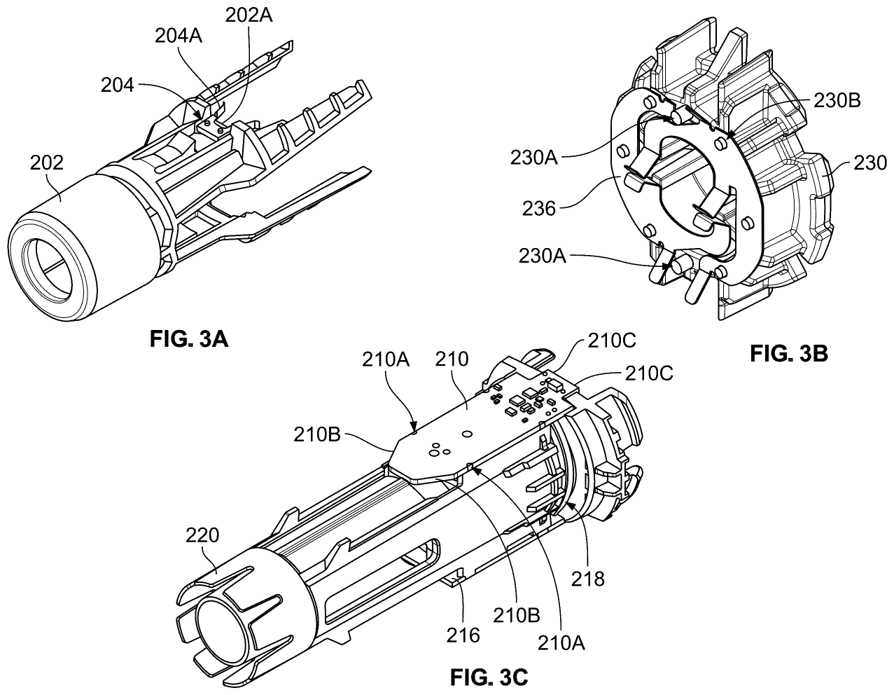

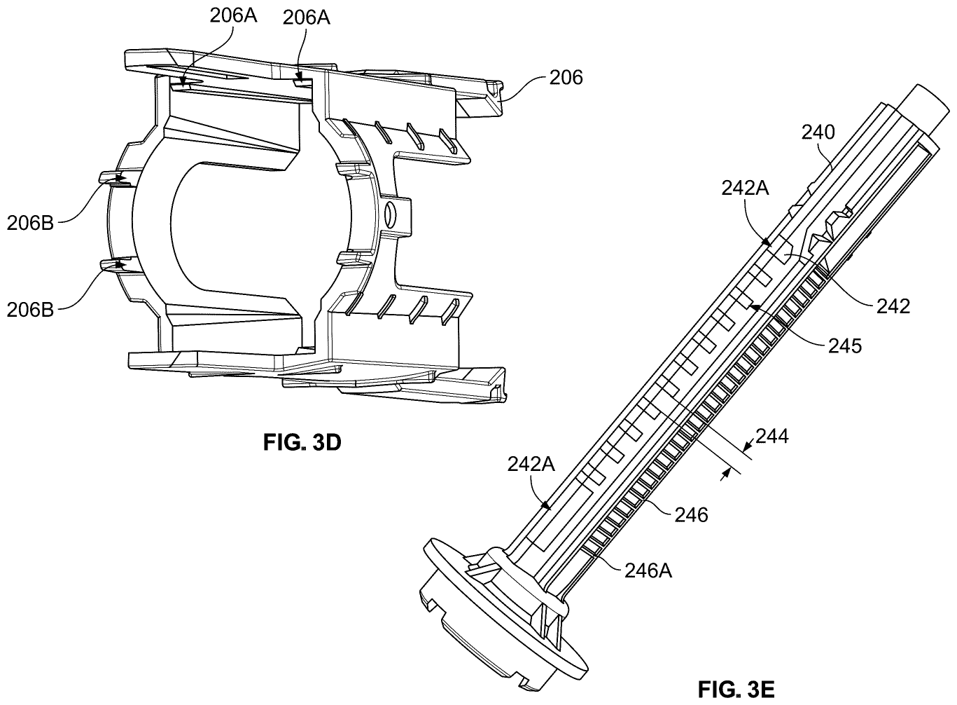

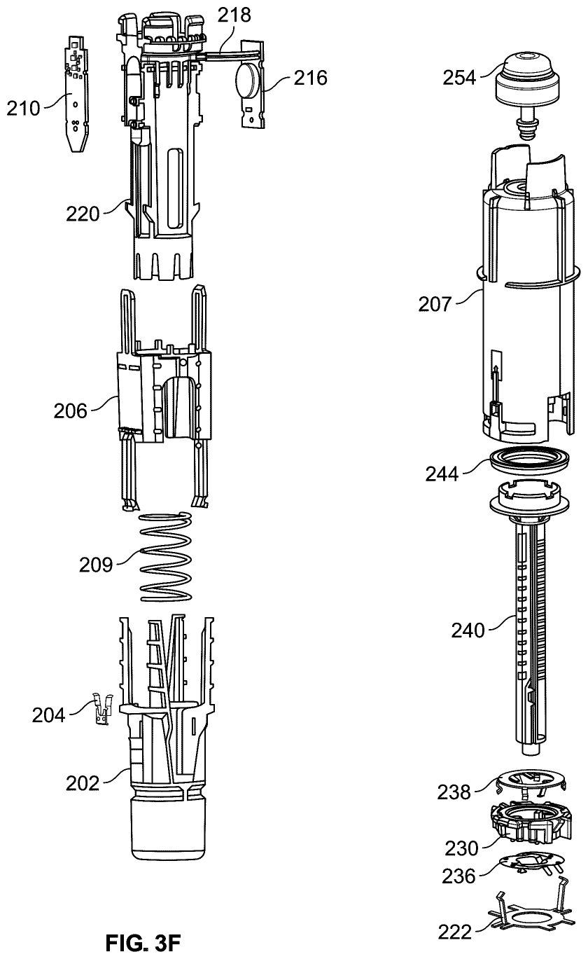

FIG. 2 is a cut-away view of an example autoinjector 200. FIGS. 3A-3E illustrate views of example components of the autoinjector 200. FIG. 3F is an exploded view of the autoinjector 200. The autoinjector 200 includes a housing 205 and a front cap 208. The front cap 208 is disposed at a distal end 203 of the housing 205. When installed on the autoinjector 200 (as shown in FIG. 2), the front cap 208 covers the needle guard 202. The user removes the front cap 208 from the autoinjector 200 prior to use of the autoinjector 200. The needle guard 202 protects the needle when the autoinjector 200 is not in use. The autoinjector 200 includes a container 212 (e.g., a syringe) within the housing 205 for holding a medicament to be injected. The container 212 may have an internal volume of 1.5-3 mL. The container 212 may have an internal volume of 1 mL, 2.25 mL, 3 mL, or 5 mL. The medicament may have a volume of 0.5-5 mL. Preferably the medicament has a volume of 1.5-3 mL. For example, the medicament may have a volume of 1.5 mL, 2.25 mL or 3 mL. The container 212 is disposed in a syringe carrier 220 and a syringe backstop 222 hold and maintain the position of the container 212 within the housing 205. A plunger is slidably disposed within the container 212. A plunger rod 240 is configured to push the plunger through the container 212 to dispense the medicament through the needle. The plunger rod 240 protrudes through a lock ring 230. The plunger rod 240 and lock ring 230 are disposed within a delivery chamber 207.

At a proximal end 201 of the autoinjector 200, a rear case 250 is coupled to the housing 205. The proximal end 201 also includes a spring 252 (e.g., an anti-rattle spring) and a gas canister assembly 254. The spring 252 is disposed between the rear case 250 and the delivery chamber 207. The spring 252 biases the delivery chamber 207 toward the distal end 203. The gas canister assembly 254 includes pressurized gas that when released provides a force acting on the plunger rod 240 to push the plunger through the container 212 in a dispensing movement to dispense the medicament. During a dispensing movement, the pressurized gas is contained in the delivery chamber 207, and a piston seal 244 reduces leakage around the plunger rod 240.

Activation of the gas canister assembly 254 can occur by a user fully depressing the needle guard 202 into the housing 205 (e.g., by pressing the needle guard 202 and the autoinjector 200 against his or her skin), such that the needle guard 202 moves the transfer sleeve 206 proximally. In turn, this causes all of the internal components (e.g., including the delivery chamber 207) to move a relatively small proximal distance against the bias of the spring 252 disposed at a proximal end 201 of the autoinjector 200. For example, the relatively small proximal distance can be a distance of 1-10 millimeters. Upon moving such a relatively small proximal distance, the gas canister assembly 254 can be activated (e.g., by movement of a firing pin that pierces an end of a gas canister within the gas canister assembly 254), thereby releasing compressed gas from within. The compressed gas can include, for example, argon, carbon dioxide, krypton, xenon, etc. Optionally, depression of the needle guard 202 can cause an internal component to contact a sensing assembly including a force sensor, such that the force sensor can measure the insertion force of the needle into the user (e.g., as described herein).

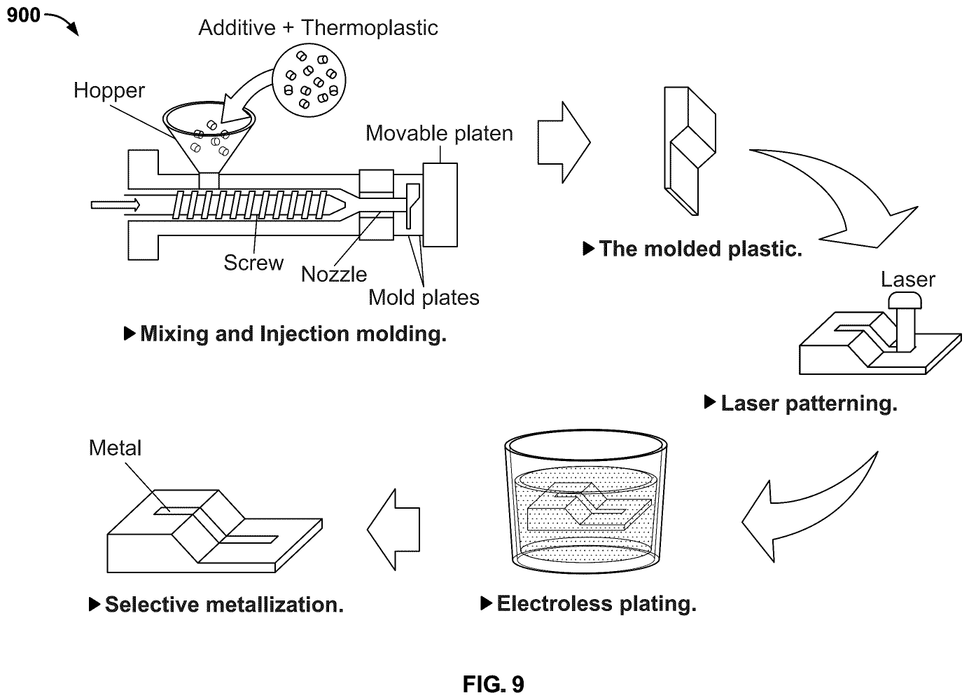

As seen in FIG. 3E, the plunger rod 240 can include one or more conductive regions (e.g., conductive contact 242) disposed along the length of the plunger rod 240 forming a dose progress track 245. The conductive contacts form a pattern that can be detected by a sensing assembly of the autoinjector 200 to determine dose progression and when the end of the dose has been reached. The pattern may include regular and irregular shapes, sizes, and/or locations of the conductive contacts and the spacings therebetween. Optionally, one or more enlarged conductive contacts 242A can be disposed near each end of the plunger rod 240 to signal the start and end of a dispense operation. Conductive contacts between the enlarged conductive contacts 242A are spaced with a spacing 244 (e.g., a regular spacing between conductive contacts). The conductive contacts can be disposed on the plunger rod 240 using laser discrete structuring (LDS) which will be described in more detail in reference to FIG. 9. Such conductive contacts can be provided in any useful manner, such as sintering, depositing, plating, coating, painting, spraying, printing, etching, patterning, annealing, or combinations thereof. Electrical contact between the conductive contact 204 of the plunger rod and a lock ring contact 236 occurs during a dispensing movement, and such electrical contact can be detected by a first sensor (e.g., a voltage sensor, resistance sensor, or other sensor configured to detect an electrical signal as the plunger rod moves).

The sensing assembly of the autoinjector 200 can include a main PCB 210 and a battery PCB 216. The main PCB 210 includes electronics to sense electrical contacts (e.g. a voltage sensor, a resistance sensor), process data, and/or communicate with an external device (e.g., a mobile device, smartphone, or tablet) through a wireless communication protocol (e.g., short range radio communication, near field communication, wireless transfer protocol, Wi-Fi). The battery PCB 216 provides power to the main PCB 210 through a connection 218 (e.g., wiring). The main PCB 210 and the battery PCB 216 are coupled to the syringe carrier 220. For example, as seen in FIG. 3C, the main PCB 210 can be heat staked to the syringe carrier 220 using one or more heat-staking features 210A. The transfer sleeve 206 can slide between the main PCB 210 and the housing 205 and between the battery PCB 216 and the housing 205. As seen in FIG. 3D, the transfer sleeve 206 can optionally include reduced thickness walls 206A to accommodate the main PCB 216. Reduced width hard stops 206B could enable the connection 218 between the battery PCB 216 and the main PCB 210.

The needle guard 202 includes a needle guard contact 204. As seen in FIG. 3A, the needle guard contact 204 can be coupled to a crossbar 202A of the needle guard 202. For example, the needle guard contact 204 can be heat staked to the crossbar 202A. The needle guard contact 204 can be formed of a single stamped metallic piece with one or more flexible arms 204A. The needle guard contact 204 is configured to contact the needle guard contact 210B on the main PCB 210 (e.g., one or more contact regions edges that are plated on the edge of the main PCB 210) when the needle guard 202 is in a retracted position. The contact between the needle guard contact 204 and the needle guard contact 210B can be detected by a second sensor (e.g., a voltage sensor, resistance sensor, or other sensor configured to detect an electrical signal as the needle guard moves). Contact between the needle guard contact 204 and the needle guard contact 210B indicate that the needle of the autoinjector 200 is exposed, and the autoinjector 200 is ready for the dosing process to begin. The location and configuration of the needle guard contact 204 can be optimized to balance exposure of the needle, insertion depth of the needle into the patient's skin, and/or dosing initiation.

A needle guard spring 209 is disposed between the needle guard 202 and the syringe carrier 220. The needle guard spring 209 biases the needle guard 202 toward the distal end 203 of the housing, causing the needle guard 202 to extend from the housing 205 and cover the needle. Retraction of the needle guard 202 compresses the needle guard spring 209. The needle guard 202 can be retracted by exerting a force on the distal end 203. Contact between the needle guard contact 204 and the needle guard contact 210B can indicate that a sufficient force has been applied to the autoinjector 200 to retract the needle guard 202 and expose the needle. Upon removing the sufficient force applied to the autoinjector 200 (e.g., after the dose is dispensed), the needle guard 202 can then extend from the housing to cover the needle.

The lock ring 230 includes lock ring contact 236. As seen in FIG. 3B, the lock ring contact 236 can be, for example, a sheet metal plate with one or more deflectable arms configured to contact the plunger rod 240. The lock ring contact 236 can be positioned on the lock ring 230 using a one or more posts 230B protruding from the lock ring 230. The lock ring contact 236 can include an opening to accommodate passage of the plunger rod 240 through that opening during a dispensing movement. When a dispensing movement is initiated, the lock ring 230 shunts toward the syringe carrier 220 with the plunger rod 240. Electrical contact between the lock ring contact 236 and lock ring contacts 210C on the main PCB 210 indicate that the dispensing movement has started. Additionally, hard stops 230A stop the lock ring 230 from travelling too far within the housing 205.

As seen in FIG. 3F, the autoinjector 200 can also include a circular clicker 238 (e.g., a ring configured to include an opening therein) disposed on a proximal side of the lock ring 230. The circular clicker 238 interfaces with the plunger rod 240. As can be seen, openings and other structural elements (e.g., contacts, stops, arms, etc.) for the lock ring contact 236, the lock ring 230, and the circular clicker 238 can be configured to minimize impeding a pathway for the dispensing movement of the plunger rod 240. As the plunger rod 240 moves through the circular clicker 238 during a dispensing movement, the circular clicker 238 emits one or more audible click sounds that can be used to track the dispensing movement. For example, the circular clicker 238 can have a deflectable protrusion configured to contact a surface of the plunger rod 240. The dispensing movement of the plunger rod 240 can deflect the deflectable protrusion to cause the circular clicker 238 to produce one or more audible clicks, vibrations or the like. A surface 246 of the plunger rod 240 can be configured to deflect the deflectable protrusion of the circular clicker 238.

The circular clicker 238 can include multiple deflectable protrusions. Where the circular clicker 238 includes multiple deflectable protrusions, the plunger rod 240 can include multiple surfaces configured to deflect the deflectable protrusions. For example, and without limitation, the plunger rod 240 can include multiple surfaces configured to deflect the deflectable protrusions, in which each surface can in turn include a plurality of ridges, bumps, projections, or other non-continuous structures along the surface to provide a ridged surface or other non-continuous surface (see discussion below). Alternatively, the circular clicker 238 may include a single deflectable protrusion. Where the circular clicker 238 includes a single deflectable protrusion, the plunger rod 240 can include a single surface 246 configured to deflect the deflectable protrusion. Any number of deflectable protrusions can be employed to produce one or more clicks (e.g., a single deflectable protrusion or multiple deflectable protrusions). In one non-limiting example, a single deflectable protrusion can be employed to reduce the possibility of interference between multiple audible clicks. In another non-limiting example, multiple deflectable protrusions can be employed to operate in sync to provide a more distinct click sound, in which interference can be minimized, e.g., by control of tolerancing and/or alignment.

The circular clicker can be a portion of the autoinjector housing. For example, the autoinjector housing can include a deflectable protrusion that contacts the plunger rod 240. The plunger rod 240 can include one or more deflectable protrusions, and the housing (or another internal component of the autoinjector) includes a surface configured to deflect the one or more deflectable protrusions.

As seen in FIG. 3E, a plunger rod 240 includes a surface 246 having one or more ridges 246A, in which a ridged surface can be configured to contact a deflectable protrusion (e.g., of an audible clicker, of a housing, or another appropriate component of the autoinjector). The ridged surface can include any appropriate number (e.g., a plurality, such as 1-100, 1-90, 1-80, 1-70, 1-60, 1-50, 1-40, 1-30, 1-20, 2-100, 2-50, 3-100, 3-50, 4-100, 4-50, 5-100, 5-50, 5-15, 8-100, 8-50, 10-100, 10-50, 10) of ridges. Each ridge of a ridged surface can deflect the deflectable protrusion to cause the deflectable protrusion to produce a signal (e.g., an audible click, a vibration, etc.) that can be sensed by a sensing assembly. For example, an example sensing output can include multiple signals, in which each signal corresponds to a ridge of a ridged surface. In turn, the number of signals can be used to track the number of ridges along the plunger rod 240 that passed by the circular clicker during a dispensing movement of the plunger rod 240. For example, a sensing assembly can use the received signals to determine that the plunger rod 240 has completed the dispensing movement after a predetermined number (e.g., a plurality, such as 1-100, 1-90, 1-80, 1-70, 1-60, 1-50, 1-40, 1-30, 1-20, 2-100, 2-50, 3-100, 3-50, 4-100, 4-50, 5-100, 5-50, 5-15, 8-100, 8-50, 10-100, 10-50, 10) of signals. The ridged surface can be any non-continuous surface configured to provide a plurality of non-continuous structures (e.g., bumps, projections, ribs, and the like upon that surface).

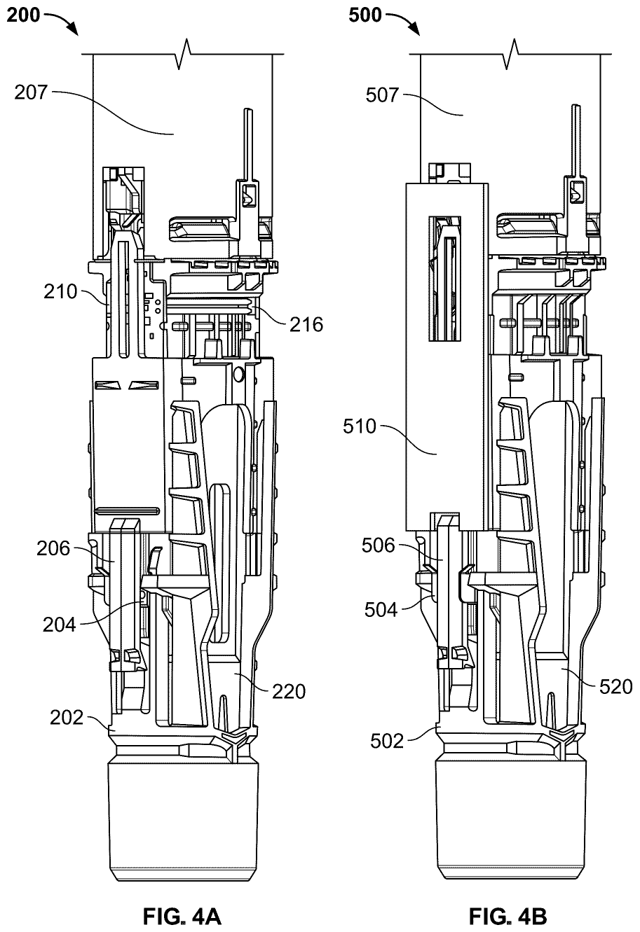

FIG. 4A illustrates a distal portion of autoinjector 200 showing the main PCB 210 and battery PCB 216 disposed on an outer surface of the syringe carrier 220, such that the main PCB 210 and battery PCB 216 are located between the syringe carrier 220 and the transfer sleeve 206 in an assembled autoinjector 200. During use, the needle guard 202 and transfer sleeve 206 move in a proximal direction (e.g., toward proximal end 201). The transfer sleeve 206 moves over the main PCB 210 and battery PCB 216 without contacting the main PCB 210 or battery PCB 216.

FIG. 4B illustrates a distal portion of another example autoinjector 500 that includes a main PCB 510 disposed on an outer surface of transfer sleeve 506. During use, the main PCB 510 can translate with the transfer sleeve 506. In some implementations, positioning the main PCB 510 on the outer surface of the transfer sleeve 506 can provide certain advantages, for example, the transfer sleeve 506 does not include sections with reduced wall thickness allowing for a stronger transfer sleeve 506 as compared with transfer sleeve 206. The syringe carrier 520 also does not include cutouts in the syringe carrier 520 to accommodate the main PCB 510. Main PCB 510 may include a battery and autoinjector 500 does not include a separate battery PCB. Further, positioning the main PCB 510 closer to the housing can enable easier removal of the main PCB 510 when disposing of the autoinjector 500. In this way, electronic components of the main PCB 510 and/or the battery 511 can be reused or disposed of properly.

As also seen in FIG. 4B, the autoinjector 500 includes a needle guard contact 504 that has been repositioned to contact an edge of the main PCB 510, which has been repositioned to the outer surface of the transfer sleeve 506. One or more modifications to structural crossbars to the needle guard 502 can allow for housing or attaching the needle guard contact 504 in a desired position.

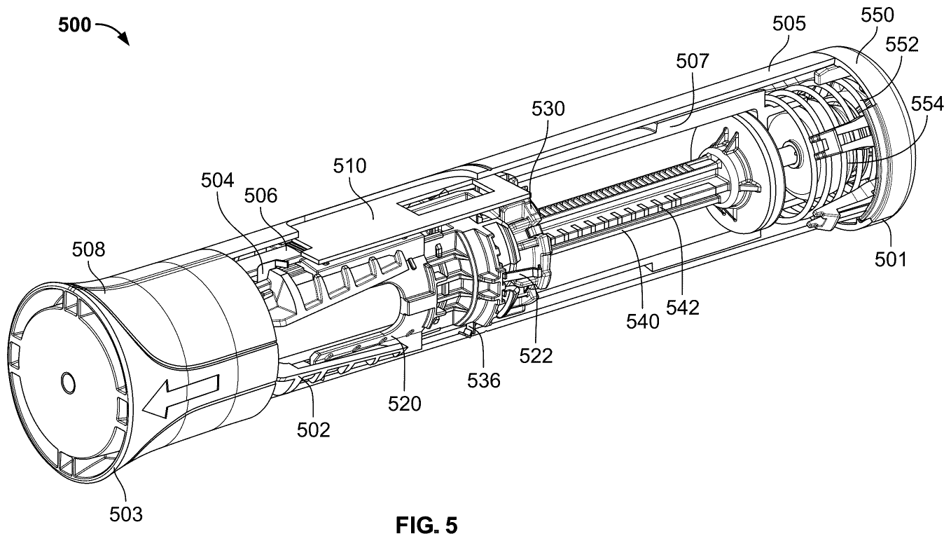

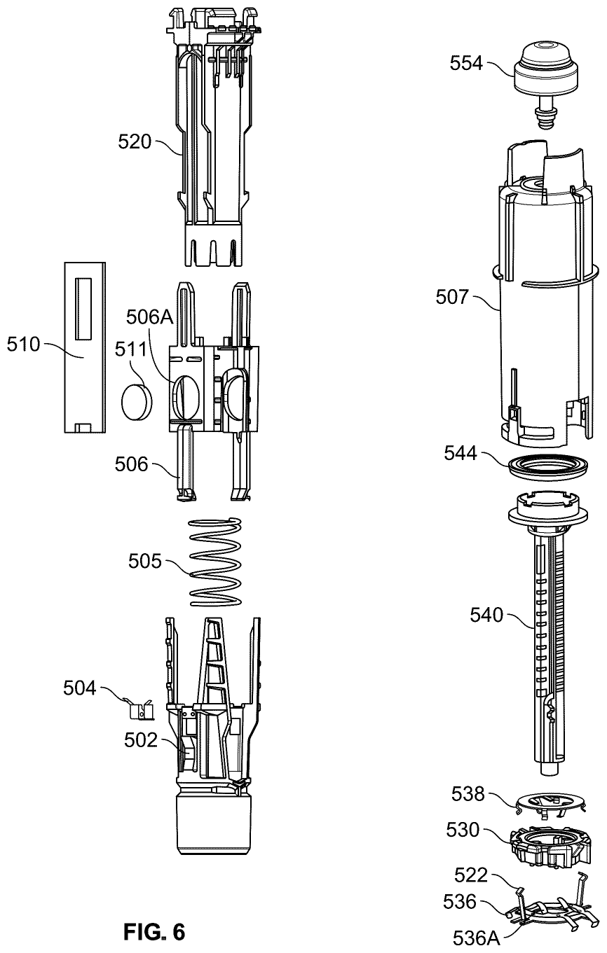

FIG. 5 is a cutaway view of autoinjector 500. FIG. 6 is an exploded view of autoinjector 500. Autoinjector 500 is substantially similar to autoinjector 200 with some differences including: the location of the main PCB 510 between the transfer sleeve 506 and the housing 505; the location of the needle guard contact 504 in view of the repositioned main PCB 510; the absence of a separate battery PCB; and the strengthened transfer sleeve 506 and syringe carrier 520, as compared with transfer sleeve 206 and syringe carrier 220. As seen in FIG. 6, the transfer sleeve 506 includes a cutout 506A to accommodate a battery 511 between the main PCB 510 and transfer sleeve 506.

At the distal end 503 of the housing 505, the autoinjector 500 includes a front cap 508 that covers the needle guard 502 when the front cap 508 is installed on the autoinjector 500. The needle guard 502 protects the needle when the autoinjector is not in use. The needle guard 502 includes a needle guard contact 504. The needle guard contact 504 comes into contact with an electrical contact on the main PCB 510 when the needle guard 502 is in a retracted position. A needle guard spring 505 is positioned between the needle guard 502 and the syringe carrier 520. The needle guard spring 505 biases the needle guard 504 toward the distal end 503. The needle guard spring 505 compresses when the needle guard 504 is pushed toward the proximal end 503 of the housing 505. The transfer sleeve 506 translates with the needle guard 505 and interfaces with the delivery chamber 507 to activate the gas canister assembly 554. A syringe backstop 522 holds the syringe carrier 520 in place within the housing 505.

The gas canister assembly 554 is positioned at the proximal end 501 between the rear case 550 and the delivery chamber 507. A spring 552 is disposed around the gas canister assembly 554. The spring 552 biases the delivery chamber 507 toward the distal end 503. When the delivery chamber 507 is moved toward the proximal end 501 a distance sufficient to activate the gas canister assembly 554, the gas canister assembly 554 releases a pressurized gas to exert a force on the plunger rod 540 to begin a dispensing movement. A piston seal 544 reduces leakage of the pressurized gas past the plunger rod 540.

A lock ring 530 is positioned between the delivery chamber 507 and the syringe carrier 522. On a proximal side, the lock ring 530 can interface with a circular clicker 538, that produces audible clicks during a dispensing movement. On a distal side, the lock ring 530 interfaces with lock ring contact 536. The lock ring contact 536 includes an opening through which the plunger rod 540 translates. The lock ring contact 536 and a syringe backstop 522 may form a subassembly, which in turn can be attached to the lock ring 530. During a dispensing movement, the lock ring 530 shunts toward the distal end 503 along with the plunger rod 540. The lock ring contact 536 includes one or more deflectable contacts 536A that are configured to contact conductive contacts 542 of the plunger rod 540. The deflectable contacts 536A may make contact at two points on the plunger rod 540. The lock ring 530 can also make electrical contact with the main PCB 510 generating an electrical signal to indicate the start of dispensing a dose of medicament.

The lock ring contact can include any number and configuration for a deflectable contact. Each deflectable contact may include a contact arm that provides a contact point with a conductive region of the plunger rod. A plurality of deflectable contacts may be employed (e.g., two, three, four, or more deflectable contacts), and each deflectable contact includes a contact arm that provides a separate contact point with a conductive region of the plunger rod. Alternatively, a deflectable contact may include a pair of contact arms (e.g., a pair including two contact arms), in which each contact arm provides a separate contact point with a conductive region of the plunger rod. One or more deflectable contacts may be disposed around an opening, in which the opening is configured to provide a travel pathway for the dispensing movement of the plunger rod. One or more deflectable contacts may be configured and/or oriented to minimize restriction of the dispensing movement of the plunger rod. In any such configuration, an electrical pathway can be provided between one or more deflectable contacts and a conduction region of the plunger rod.

The main PCB 510 forms a sensing assembly and includes at least one sensor. For example, the main PCB 510 can include one or more voltage sensors to sense when electrical contact is made (e.g., between the needle guard contact 504 and the main PCB 510 and/or between the conductive contact 542 of the plunger rod 540 and the lock ring contact 536). Alternatively, or additionally, the main PCB 510 includes one or more resistance sensors. The main PCB 510 may include a thermistor to measure an ambient temperature in the area in proximity to the container that includes the medicament. The main PCB 510 may include a force sensor to measure an insertion force to insert a needle into a user.

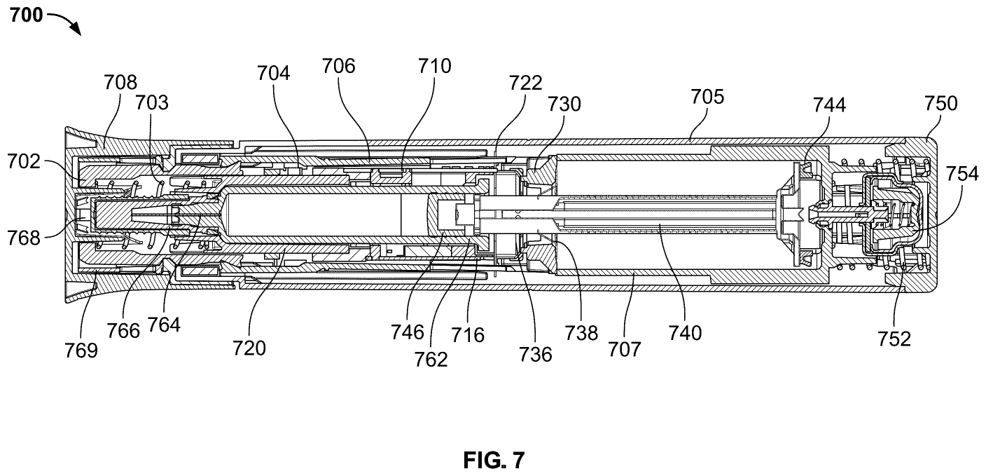

FIG. 7 is a cross-section view of yet another example autoinjector 700. The autoinjector 700 includes a housing 705 and front cap 708 coupled to a distal end of the housing 705. The front cap 708 includes a needle shield remover 768 that can remove a needle shield 766 from a needle 764 when the front cap 708 is removed from the housing 705. The front cap 708 also includes an anti-drop ring 769 to reduce the likelihood of accidental removal of the front cap 708 due to the autoinjector 700 being dropped by a user.

The needle 764 is connected to container 762. The needle shield 766 and needle guard 702 protect the needle 764 prior to use of the autoinjector 700. The needle guard 702 is biased away from the container 762 by the needle guard spring 705. The needle guard 702 includes a needle guard contact 704 that can contact the main PCB 710 during use of the autoinjector 700 to indicate that the needle 764 is inserted to an appropriate depth in the patient.

The container 764 can include a medicament to be injected into a patient. The container 764 may have an internal volume of 1.5-3 mL. The container 764 may have an internal volume of 1 mL, 2.25 mL, 3 mL, or 5 mL. The medicament may have a volume of 0.5-5 mL. Preferably the medicament has a volume of 1.5-3 mL. For example, the medicament may have a volume of 1.5 mL, 2.25 mL or 3 mL. The container 764 is held in place within the autoinjector 700 by syringe carrier 720. A syringe backstop 722 holds the syringe carrier 720 in place restricting movement of the syringe carrier 720 toward the rear case 750.

A transfer sleeve 706 is positioned around the syringe carrier 722. The transfer sleeve 706 interfaces between the needle guard 702 and the delivery chamber 707. The main PCB 710 and a battery PCB 716 are located between the transfer sleeve 706 and the syringe carrier 720. Alternatively, the battery PCB may be absent, and the main PCB may optionally be located between the transfer sleeve 706 and the housing 705.

The plunger rod 740 protrudes through a lock ring 730 to exert a force on the plunger 746 that is slidably disposed within the container 762 to dispense the contents of the container 762. A lock ring contact 736 is disposed between the lock ring 730 and syringe carrier 720 on a distal side of the lock ring 730, and a circular clicker 738 is disposed on a proximal side of the lock ring 730.

The delivery chamber 707 is disposed between the lock ring 730 and the gas canister assembly 754. The delivery chamber 707 includes the plunger rod 740. The piston seal 744 forms a seal between the plunger rod 740 and sides of the delivery chamber 707 to reduce leakage of the pressurized gas from the gas canister assembly 754 past the plunger rod 740. The gas canister assembly 754 is disposed between the delivery chamber 707 and the rear case 750. A spring 752 is disposed around the gas canister assembly 754. The spring 752 biases the delivery chamber 707 toward the needle 764.

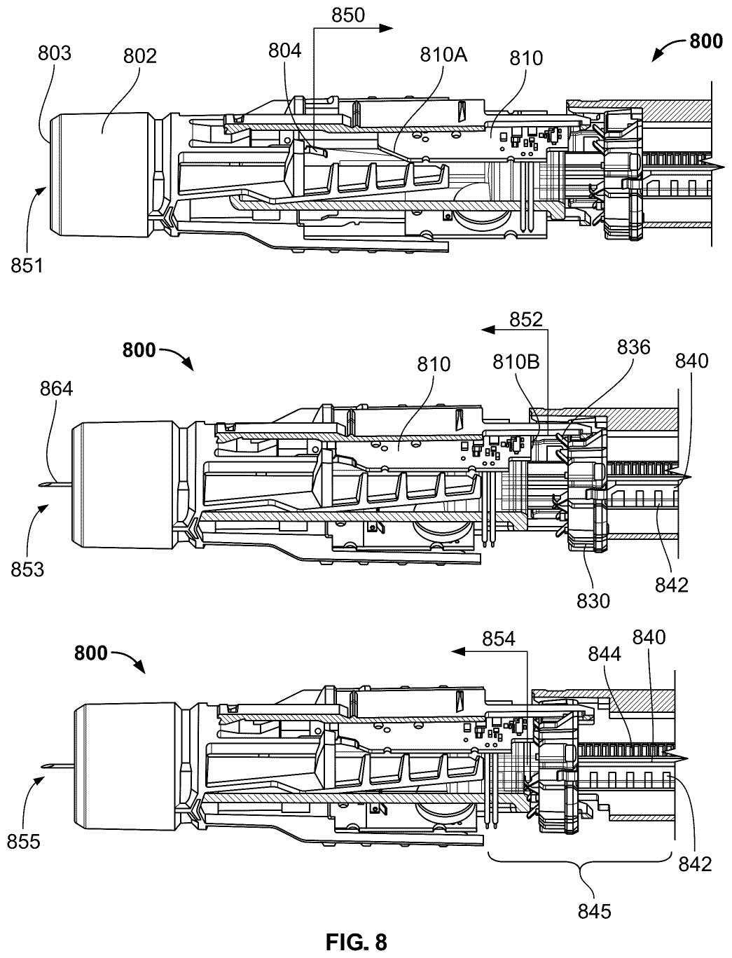

FIG. 8 illustrates a dispensing movement of an example autoinjector 800. At position 851, a force is exerted against the distal end 803 of the needle guard 802. The needle guard contact 804 moves 850 toward the main PCB 810. At position 853, the needle 864 is exposed though the needle guard 802. The needle guard contact 804 is in electrical contact with needle guard contact 810A of the main PCB 810. The contact between the needle guard contact 804 and the needle guard contact 810A indicates that the needle 864 is exposed a specified distance beyond the distal end 803 of the needle guard 802 (e.g., the needle 864 is inserted to an appropriate depth in a patient). The movement of the needle guard 802 activates the gas canister assembly to begin releasing pressurized gas into the delivery chamber. The lock ring 830, lock ring contact 836, and plunger rod 840 translate 852 toward the distal end 803. The lock ring contact 836 makes electrical contact with a lock ring contact 810B on the main PCB 810 indicating that the dispensing movement has started. At position 855, the plunger rod 840 slides 854 through the lock ring 830. Conductive contacts 842 on the dose progress track 845 of the plunger rod 840 make contact with the lock ring contact 836. One or more conductive contacts 842 can be configured to track certain events during dose progression. For example, and without limitation, a first conductive contact can be configured to indicate dose start of the medicament, and/or a last conductive contact can be configured to indicate dose end of the medicament. Optionally, the first and last conductive contacts can be different than other conductive contact(s) on the dose progress track. For example, and without limitation, the first and last conductive contacts can have differing shape, material, spacing, and the like to provide a differing electrical signal (e.g., an electrical signal of differing voltage, resistance, amplitude, frequency, duration of signal, duration of time between signals, etc.).

Passage of the conductive contacts 842 through the lock ring 830 generates multiple electrical signals that can be used to track the dose progress of the dispensing movement. The plunger rod 840 also includes ridges (e.g., ridge 844) that can interface with an audible clicker (e.g., by way of a circular clicker in proximity to the lock ring, as described herein) to generate audible clicks as the dose progresses. The audible clicks form an alternate way (or an additional way with the electrical signals from the conductive contacts 842) of tracking dose progress during the dispensing movement.

FIG. 9 is an example process 900 for forming the conductive contacts on the plunger rod of an autoinjector using laser discrete structuring (LDS). The plunger rod is formed by mixing a thermoplastic and additives in a hopper of an injection molding machine. The thermoplastic and additives are fed by a screw through a nozzle into mold plates to form the plunger rod. A movable piston can be moved to release the molded plastic (e.g., plunger rod) from the mold plates. The molded plastic then goes through a laser patterning step where a laser etches a desired pattern on the plastic. For example, the laser etches the desired pattern of conductive contacts on the plunger rod. The molded plastic then goes through an electroless plating process where the patterned regions on the molded plastic are metallized forming selectively metallized portions of the molded plastic. Other manufacturing and fabrication processes may be employed to form the plunger rod, and other patterning and deposition processes may be employed to form the conductive contact on a surface of the plunger rod.

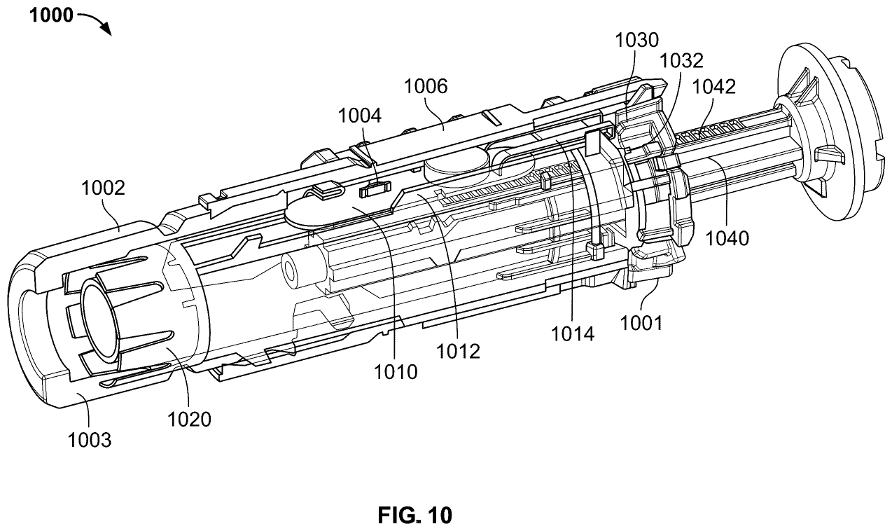

FIGS. 10-13C illustrate another example autoinjector 1000 having a distal end 1003 and a proximal end 1001. The autoinjector 1000 is substantially similar to autoinjectors 100, 200, 500, 700 and 800. Some differences between the autoinjector 1000 and the previously described autoinjectors include differences in the main PCB 1010 and the lock ring 1030, as well as the inclusion of a needle guard switch 1004, thermistor 1012, lock ring PCB 1032, and connector 1014.

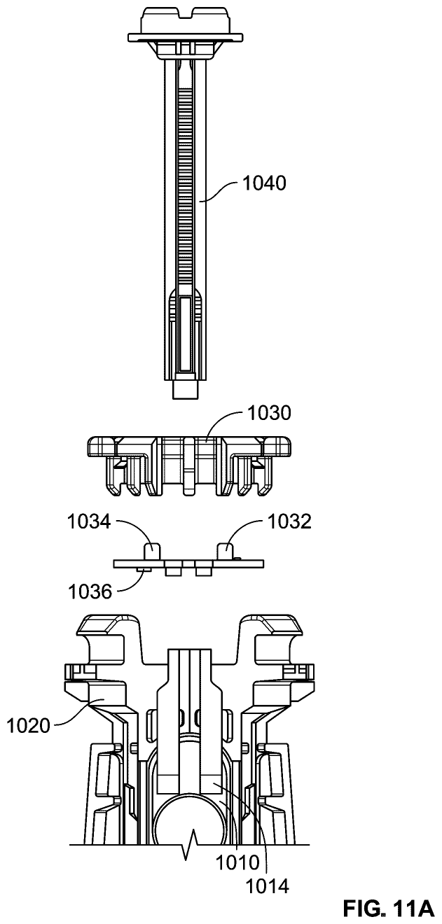

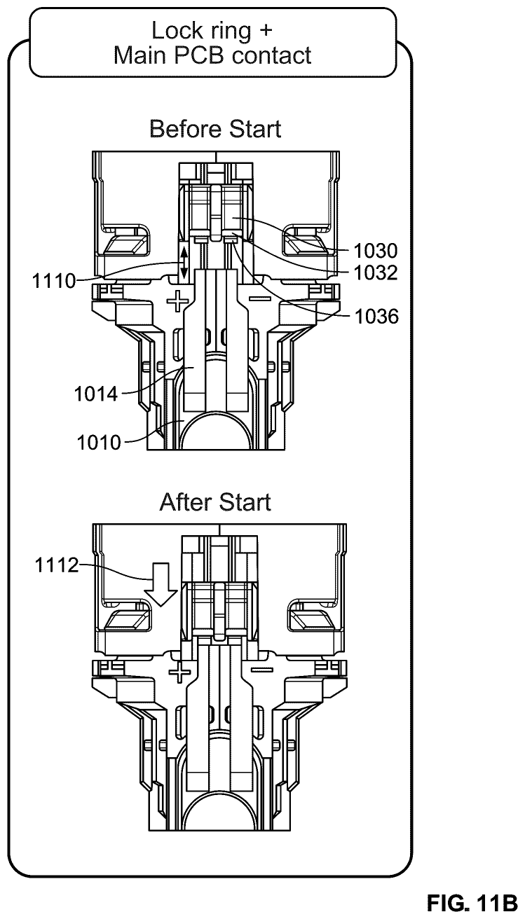

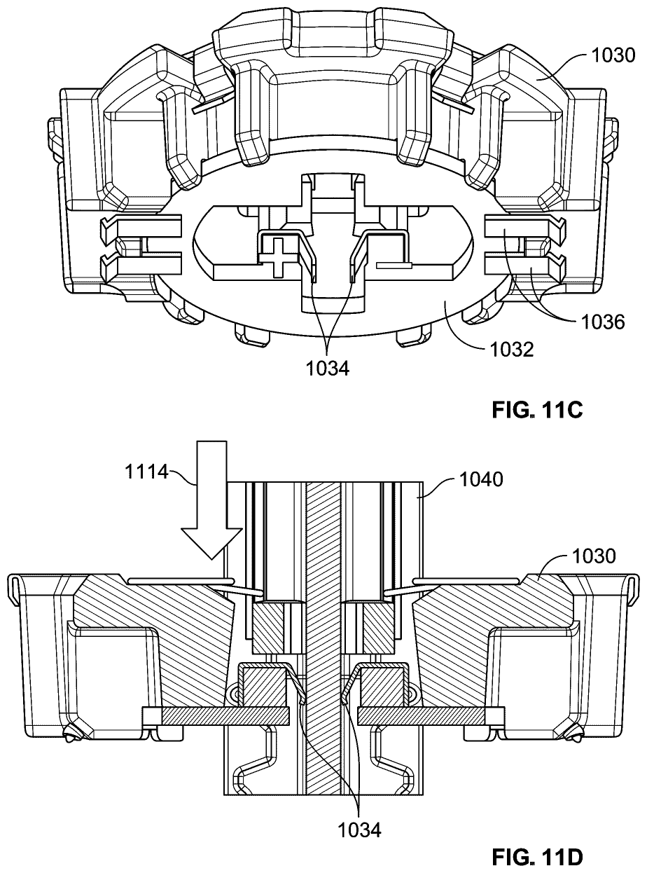

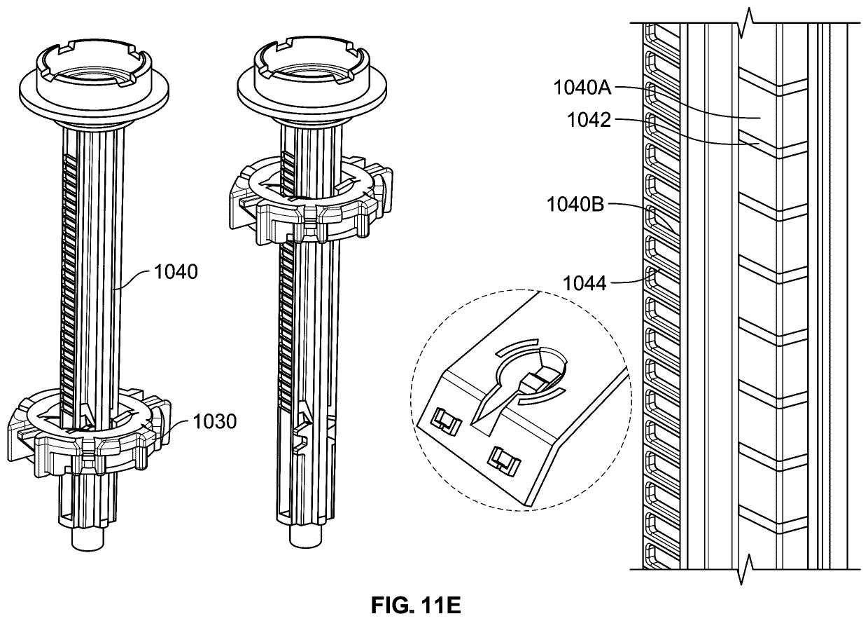

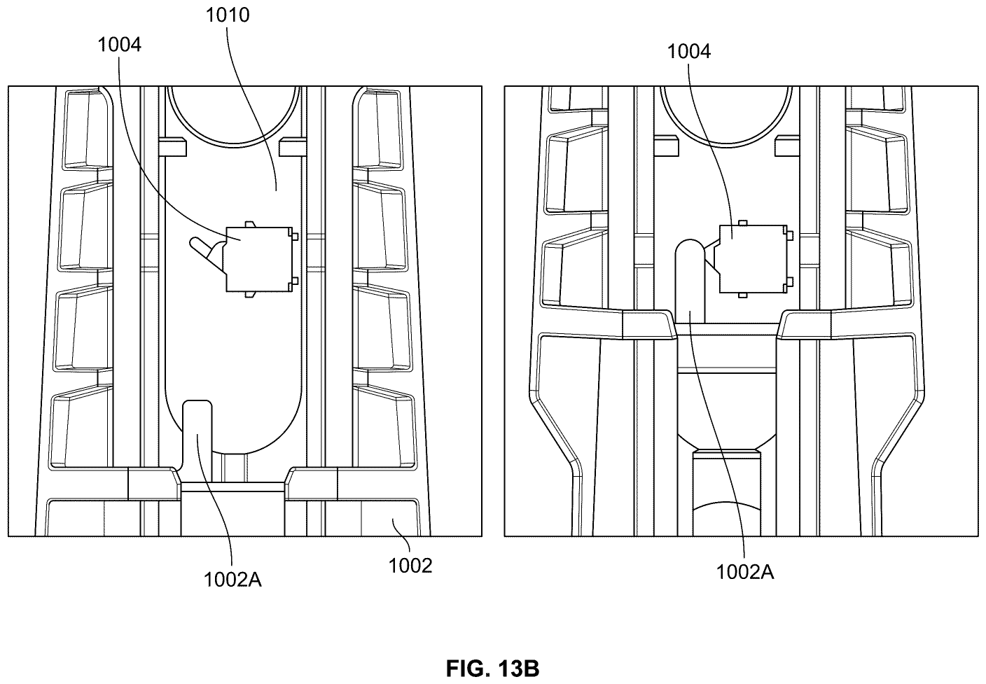

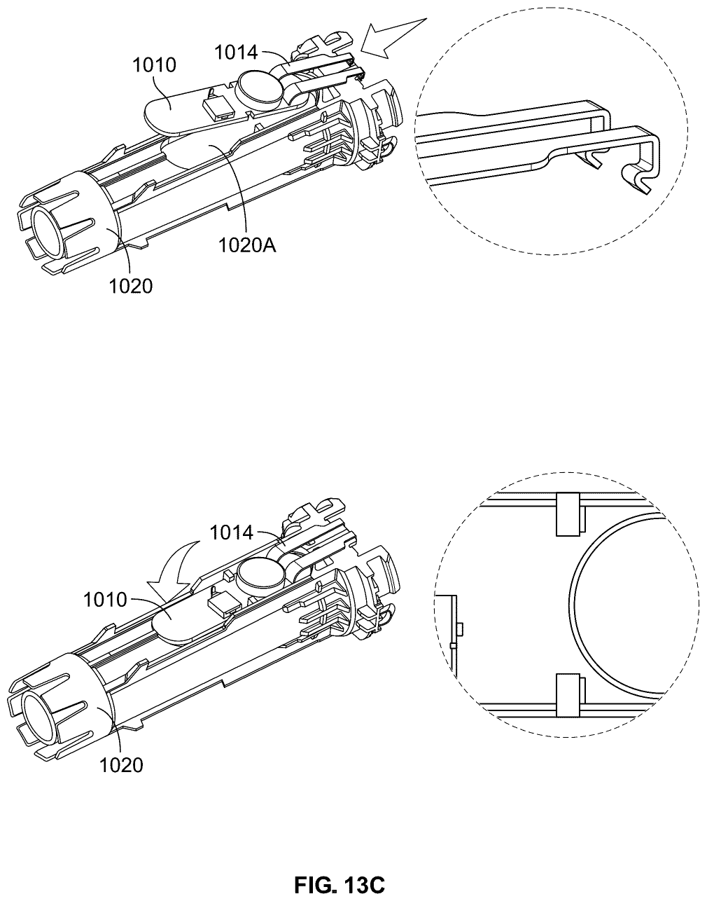

The main PCB 1010 is disposed between the syringe carrier 1020 and the transfer sleeve 1006. The syringe carrier 1020 includes a syringe carrier cutout 1020A to accommodate the main PCB 1010 (as seen in FIG. 13A). The main PCB 1010 is held in place with one or more press fit joints within the syringe carrier 1020. The main PCB 1010 includes the needle guard switch 1004 and thermistor 1012. As seen in FIG. 11A, a main PCB connector 1014 performs at least two functions. The connector 1014 attaches the main PCB 1010 to the syringe carrier 1020. Also, the connector 1014 forms electrical contact surfaces to contact the first contact 1034 and second contact 1036 on the lock ring PCB 1032 (as seen in FIG. 13C).

The lock ring PCB 1032 is attached to a distal side of the lock ring 1030, such that the lock ring PCB 1032 is positioned between the lock ring 1030 and the syringe carrier 1020. Before the start of a dispensing movement (as seen in FIG. 11B), there is a gap 1110 between the main PCB connectors 1014 (of the main PCB 1010) and the first contact 1034 and second contact 1036 (of the lock ring PCB 1032). After the dispensing movement has started, the lock ring 1030 has translated 1112 toward the syringe carrier 1020 and the main PCB 1010 causing electrical contact between the lock ring PCB 1032 and the connectors 1014 of the main PCB 1010. The electrical contact can indicate that the dispensing operation has started. The lock ring PCB can be replaced with a lock ring contact (e.g. as described herein).

As the dose progresses, conductive contacts 1042 on the plunger rod 1040 form electrical contacts with the first contact 1034 of the lock ring PCB 1032 to track the progress of the dispensing movement 1114 (as seen in FIG. 11D). The plunger rod 1040 may include conductive contacts 1042 on a first surface 1040A of the plunger rod 1040. A second surface 1040B of the plunger rod 1040 includes ridges 1044 (as seen in FIG. 11E). The ridges 1044 can deflect portions of an audible clicker that emits an audible click for each ridge that passes the audible clicker disposed in proximity to the lock ring 1030. The audible clicker can form a secondary indication of the dose progress.

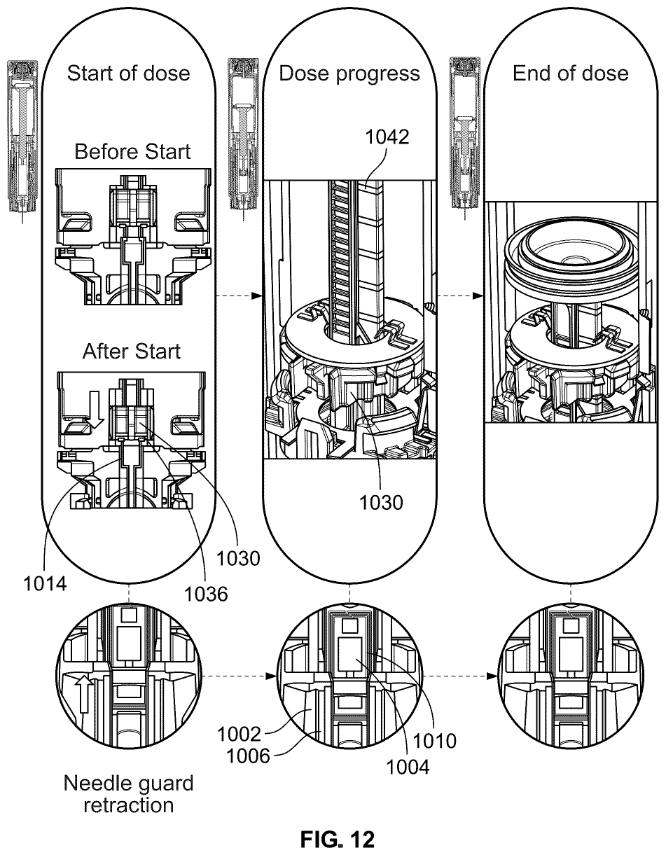

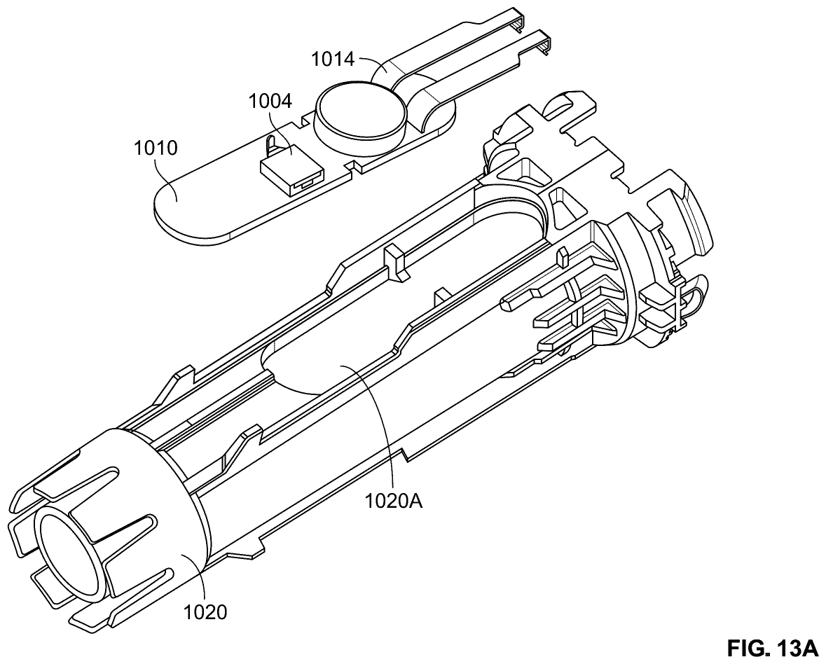

Prior to the start of dosing, the needle guard switch 1004 can be in a first position indicating that the needle guard 1002 has not been retracted (as seen in FIG. 12). When the needle guard 1002 is retracted, a needle guard feature 1002A actuates the needle guard switch 1004 to a second position indicating that the needle guard 1002 has been retracted a sufficient distance to start the dispensing movement (as seen in FIG. 13B).

The thermistor 1012 on the main PCB 1010 can be used to measure (indirectly or directly) the temperature (e.g., an approximate temperature) of a medicament included in the container in the syringe carrier 1020. Direct temperature of the medicament may impact sterility of the medicament. Indirect temperature reading of the medicament (e.g., by detecting an ambient temperature within the autoinjector and/or in proximity to the medicament) may be sufficient for dose initiation. A reading from the thermistor 1012 can be used to determine that the medicament is at a specified temperature to be injected into a patient. For example, the thermistor 1012 can measure the ambient temperature in the region near the syringe carrier and the temperature of the medicament can be inferred based on the ambient temperature near the syringe carrier. Optionally, the main PCB 1010 can restrict dispensing of the medicament until the thermistor 1012 indicates that an appropriate temperature (e.g., ambient temperature) of the medicament has been reached.

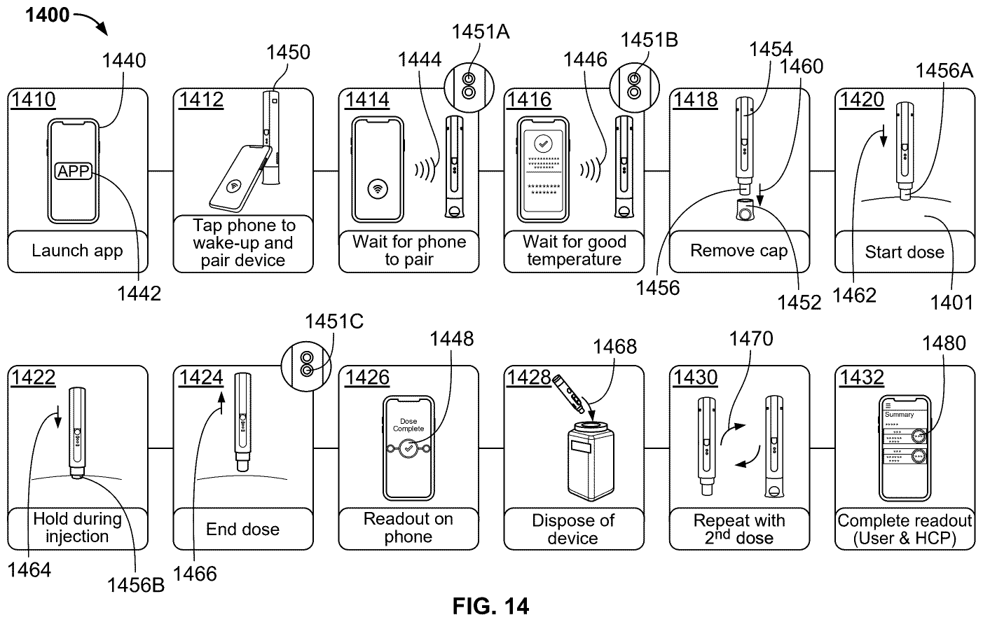

FIG. 14 illustrates an example method 1400 of using an autoinjector 1450. The autoinjector can be similar to, e.g., the autoinjectors 100, 200, 500, 700, 800, and 1000 described above. In the method 1400, the autoinjector 1450 is in communication with a mobile device 1440. For example, the autoinjector can be in communication with the mobile device 1440 via a wireless transfer protocol module or near field communication protocol (see discussion above). In step 1410, the method 1400 includes launching an app 1442 and/or website on the mobile device 1440. In step 1412, the method 1400 includes pressing a button to wake up the electronics in the autoinjector 1450. For example, such a button can be included on the housing of the autoinjector 1450. The button can be included on the app 1442 and/or website on the mobile device 1440. The button may not be activated, but, optionally, activation of a force sensor or electrical contact between elements in the autoinjector can wake up the electronics in the autoinjector. The electronics in the autoinjector can include, e.g., a PCB sub-assembly, a voltage sensor, a resistance sensor, a temperature sensor (e.g., a thermistor), a force sensor (e.g., a force sensitive resistor, a load cell, a strain gauge, a force sense capacitor), a vibration sensor (e.g., an accelerometer, a microphone (e.g., a contact microphone or an air microphone), a displacement sensor, a velocity sensor)), a switch (e.g., a needle guard switch), and the like. Optionally, the voltage/resistance sensor can sense a movement of a needle guard and/or a dispensing movement of a plunger rod that contacts a conductive contact or an electrical contact, as discussed above; the temperature sensor can sense a temperature in proximity to the medicament; the force sensor can sense an insertion force used to insert the needle of the autoinjector a sufficient distance into the patient; and/or the vibration sensor can sense a dispensing movement of a plunger rod that contacts an audible clicker, as discussed above.

In step 1414, the mobile device 1440 pairs 1444 with the autoinjector 1450. Optionally, an indicator light 1451A may indicate a status of the autoinjector 1450. In step 1416, when turned on, the temperature sensor measures a temperature, and the wireless transfer protocol module can communicate 1446 the temperature to the mobile device 1440. The temperature sensor can provide any number and type of measurements, including a temperature reading or a rate of change of temperature (e.g., 0.01-0.2° C./minute, 0.01-0.1° C./minute, 0.02-0.2° C./minute, 0.02-0.1° C./minute, about 0.05° C./minute, etc.) determined by readings determined over a time interval (e.g., a time interval of one, two, three, four, or five minutes, in which measurements can be taken every 10, 20, 30, 45, 60, or more seconds). The temperature may be an ambient temperature determined within the autoinjector. While not a direct measurement, the ambient temperature can be used to infer the temperature of the medicament. The temperature may be of a medicament contained within the autoinjector. The user can wait until the medicament is at an appropriate temperature for injection. For example, in many cases, users keep autoinjectors in a refrigerator or other chilled location. After the autoinjector is removed from the refrigerator, the medicament warms up to an appropriate temperature for injection (e.g., 5-10° C., 5-20° C., 5-30° C., 10-20° C., 10-30° C., 20-30° C., less than 30° C., etc.). An indicator 1451B on the autoinjector can indicate that the appropriate temperature has been reached.

When the medicament is at an appropriate temperature, the autoinjector 1452 is ready for use. In step 1418, the user can remove 1460 a front cap 1452 of the ready autoinjector 1454. Removing the cap 1452 of the autoinjector 1454 reveals the needle guard 1456 so that the needle can be inserted into the patient (see discussion below). In step 1420, the user brings 1462 the needle guard 1456 into contact with his or her skin 1401 to start the dose. The needle guard 1456 is initially in an extended position 1456A where the needle is covered. At step 1422, the user inserts 1464 the needle by pressing the autoinjector further into the skin. Inserting the needle into the patient's skin moves the needle guard 1456 into a retracted position 1456B. A time delay may be present between a time for inserting the needle into the skin and a time for beginning the dose of medicament. The internal components of the autoinjector inject the medicament into the user when the user inserts the needle into his or her skin (see discussion below). The user holds the autoinjector in place during the injection. In step 1424, the dose ends after the medicament has been injected into the user and the autoinjector is removed 1466 from the patient. An indicator 1451C on the autoinjector can indicate that the dose is complete.

In step 1426, after the dose ends, information about the dose can be displayed 1448 on the mobile device. For example, information can include whether the dose was successfully injected, the time of the delivery, the amount of medicament delivered, a likely error in delivery (e.g., early lift off, shallow delivery, etc.). The information can be determined based on received signals from, e.g., the force sensor, the temperature sensor, the vibration sensor, the mobile device.

In step 1428, the user can dispose 1468 of the autoinjector. The user can dispose of the entire autoinjector. Optionally, the user can remove the electronics from the autoinjector. In some non-limiting examples, the removed electronics can be disposed (e.g., to an appropriate waste stream). In other non-limiting examples, the removed electronics can be returned to the manufacturer for possible reassignment in a controlled manner. In step 1430, the user can perform an injection with a second autoinjector by performing 1470 the portions of the method 1400 described above.

In step 1432, the mobile device and/or another device, e.g., a device of a healthcare provider, can provide a full readout of information 1480 from the one or more injections performed by the user. For example, the full readout of information can include information about each of the injections performed by the user, including whether each dose was successfully injected, the time of the delivery, the amount of medicament delivered, etc.

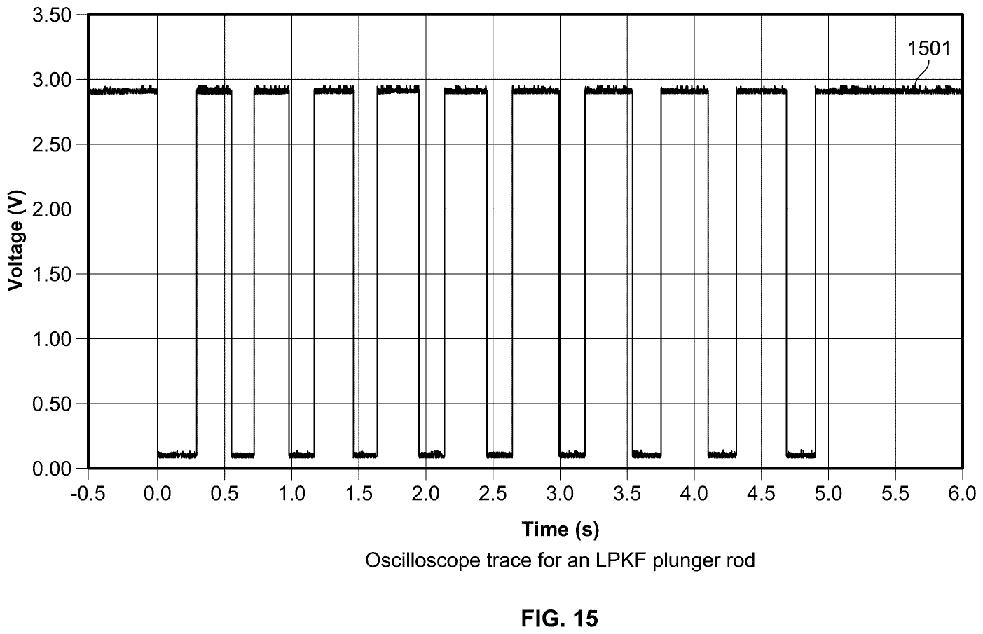

FIG. 15 is a plot of an example electrical signal 1501 generated by an autoinjector indicating dose progress of the autoinjector during a dispensing movement of the autoinjector. High voltage signals (e.g., approximately 3 V) indicate contact between conductive contacts on the plunger rod and the lock ring contact. The dose begins at time equal to zero seconds. The spacing between the square pulses in the electrical signal 1501 depends on the speed of the dispensing movement and the spacing between consecutive conductive contacts. The autoinjector can be configured to detect a certain number of pulses to determine that the injection has been completed. The length of each pulse, the signal strength of each pulse, and/or the length of spacing between pulses can be constant or may be varied. As seen in FIG. 15, at 5 seconds, a longer duration high voltage pulse is detected indicating that a larger conductive contact (e.g., positioned at the end of the plunger rod) has been detected indicating an end of the dose. Optionally, if the autoinjector does not detect the end of dispensing movement signal, the autoinjector raises an error message that the dosing was incomplete. The error message can be generated and displayed by a connected device (e.g., a mobile device).

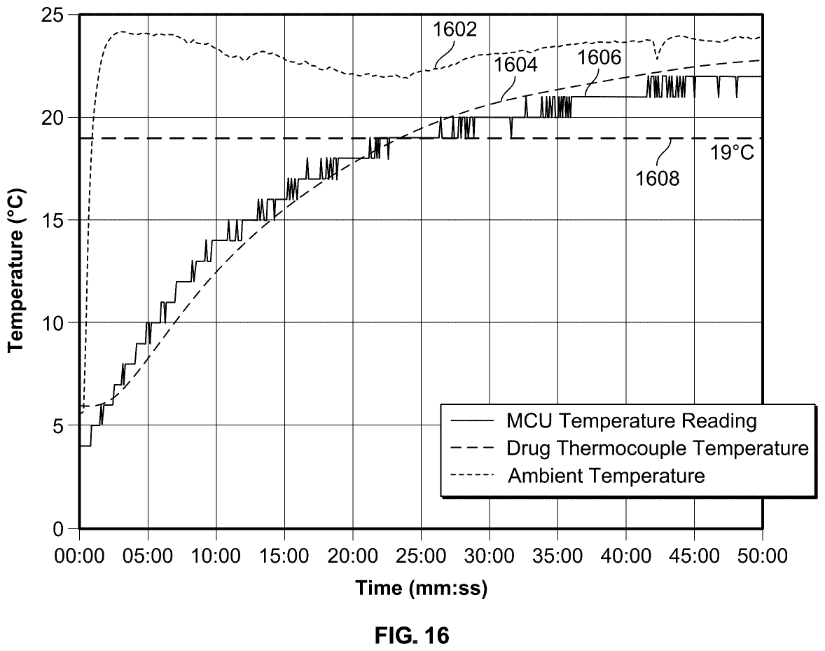

FIG. 16 is plot of temperature versus time. Three temperature traces are shown, ambient temperature 1602, temperature of a drug 1604 as measured by a thermocouple inserted into the drug, and a temperature reading 1606 from a micro-control unit (MCU) with a thermistor. A threshold injection temperature 1608 of 19° C. is also shown. When the drug is removed from a cooled location (e.g., a refrigerator), the ambient temperature quickly rises to normal room temperature. The MCU temperature reading 1606 tracks the temperature of the drug 1604 reasonably well indicating that the MCU temperature reading 1606 is an adequate proxy for the temperature of the drug 1604.

As used herein, the term “medicament” refers to a pharmaceutical formulation containing at least one active pharmaceutical ingredient (API) which is formulated for administration via injection (e.g., a liquid formulation). The API may be a compound which acts as an anti-viral or has anti-viral properties. The anti-viral API may be a compound that can treat or prevent viral disease such as HIV infection, hepatitis, or other viral disease. The anti-viral API of the medicament may treat or prevent HIV infection alone or in combination with other drugs.

The API may be an anti-HIV drug such as a capsid inhibitor (e.g., an antiretroviral drug that targets the capsid protein shell of viruses). Examples of suitable capsid inhibitors can be found, for example, in U.S. Pat. Nos. 9,951,043; 10,071,985; 11,944,611; 11,787,825; and 12,084,467; and U.S. Publication No.: 20230212148.

The capsid inhibitor may be lenacapavir sodium (SUNLENCA). As used herein, “lenacapavir” (or LEN) refers to N—((S)-1-(3-(4-chloro-3-(methylsulfonamido)-1-(2,2,2-trifluoroethyl)-1H-indazol-7-yl)-6-(3-methyl-3-(methylsulfonyl) but-1-yn-1-yl) pyridin-2-yl)-2-(3,5-difluorophenyl)ethyl)-2-((3bS,4aR)-5,5-difluoro-3-(trifluoromethyl)-3b,4,4a,5-tetrahydro-1H-cyclopropa[3,4]cyclopenta[1,2-c]pyrazol-1-yl) acetamide.

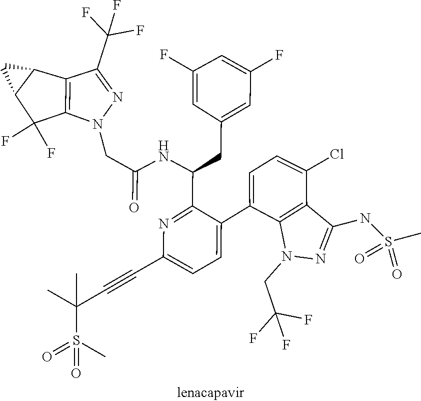 [SMILES: CC(C)(C#Cc1ccc(-c2ccc(Cl)c3c(NS(C)(=O)=O)nn(CC(F)(F)F)c23)c([C@H](Cc2cc(F)cc(F)c2)NC(=O)Cn2nc(C(F)(F)F)c3c2C(F)(F)[C@@H]2C[C@H]32)n1)S(C)(=O)=O]

Synthesis and characterization of lenacapavir, and salts thereof, are described, for example, in US patent publications US20180051005 and US20190300505. Various forms and/or uses of lenacapavir are disclosed, for example, in US20190083478, US 20190084963, US20200038389A1, and US20210188815.

The capsid inhibitor may be lenacapavir, or a pharmaceutically acceptable salt thereof. The capsid inhibitor may be lenacapavir sodium. The capsid inhibitor may be lenacapavir (i.e., the free acid form of lenacapavir).

The present disclosure further includes medicaments (i.e., pharmaceutical compositions) comprising a capsid inhibitor provided herein, or a pharmaceutically acceptable salt thereof, and at least one pharmaceutically acceptable carrier. As used herein, “pharmaceutically acceptable carrier” is meant to refer to any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier which has been approved by the United States Food and Drug Administration as being acceptable for use in humans or domestic animals.

Pharmaceutical compositions of the disclosure are formulated so as to allow the active ingredients contained therein to be bioavailable upon administration of the composition to a patient. Compositions that will be administered to a patient take the form of one or more dosage units. Actual methods of preparing such dosage forms are known, or will be apparent, to those skilled in this art; for example, see Remington: The Science and Practice of Pharmacy, 20th Edition (Philadelphia College of Pharmacy and Science, 2000). The composition to be administered will, in any event, contain a therapeutically effective amount of the API, or a pharmaceutically acceptable salt thereof (e.g., for treatment or prevention of an HIV infection or reducing the risk of acquiring HIV).

Examples of suitable excipients are well known to the person skilled in the art of parenteral formulations and can be found, for example, in the Handbook of Pharmaceutical Excipients (eds. Rowe, Sheskey & Quinn), 6th edition 2009.

Examples of excipients in a parenteral formulation (for example, a subcutaneous or intramuscular formulation) include polyethylene glycol. In general, polyethylene glycol (PEG) is a polyether having a general formula H—(O—CH₂—CH₂)ₙ—OH. The PEG may be “capped” by an alkyl group. Optionally, the capped PEG may be of the formula alkyl-(O—CH₂—CH₂)ₙ—O-alkyl (for example, CH₃—(O—CH₂—CH₂)ₙ—OCH₃). The pharmaceutical compositions of the present disclosure can include PEG having an average molecular weight of about 100 to about 1000. The average molecular weight of PEG within the pharmaceutical composition may be about 100 to about 800. The average molecular weight of PEG within the pharmaceutical composition may be about 200 to about 600. The average molecular weight of PEG within the pharmaceutical composition may be about 400. The average molecular weight of PEG within the pharmaceutical composition may be about 300. The average molecular weight of PEG within the pharmaceutical composition may be about 200. In some embodiments of the pharmaceutical composition, different molecular weight PEG can be combined to obtain a desired property or properties (for example, viscosity). Specific examples of PEG include, but are not limited to, PEG 100, PEG 200, PEG 300, PEG 400, PEG 500, and PEG 600. PEG 100, for example, refers to a polyethylene glycol with an average molecular weight of about 100.

The pharmaceutical compositions of the present disclosure can be in the form of a sterile injectable preparation, such as a solution or sterile injectable aqueous or oleaginous suspension. This suspension may be formulated according to the known art using those suitable dispersing or wetting agents and suspending agents which have been mentioned herein. The sterile injectable preparation may also be a sterile injectable solution or suspension in a non-toxic parenterally acceptable diluent or solvent, such as a solution in 1,3-butane-diol or prepared as a lyophilized powder. Among the acceptable vehicles and solvents that can be employed are water, Ringer's solution, and isotonic sodium chloride solution. In addition, sterile fixed oils can conventionally be employed as a solvent or suspending medium. For this purpose, any bland fixed oil can be employed, including synthetic mono- or diglycerides. In addition, fatty acids such as oleic acid can likewise be used in the preparation of injectables.

Example formulations suitable for use with the systems and devices of the present application can be found, for example, in U.S. Pat. No. 11,807,625.

The pharmaceutical composition may comprise the API (e.g., lenacapavir or lenacapavir sodium) at a concentration of about 10 mg/mL to about 600 mg/mL. The pharmaceutical composition may comprise the API (e.g., lenacapavir or lenacapavir sodium) at a concentration of about 100 mg/mL to about 400 mg/mL. The pharmaceutical composition may comprise the API (e.g., lenacapavir or lenacapavir sodium) at a concentration of about 250 mg/mL to about 350 mg/mL. The pharmaceutical composition may comprise the API (e.g., lenacapavir or lenacapavir sodium) at a concentration of about 300 mg/mL to about 325 mg/mL. The pharmaceutical composition may comprise the API (e.g., lenacapavir or lenacapavir sodium) at a concentration of about 305 mg/mL to about 310 mg/mL. The pharmaceutical composition may comprise the API (e.g., lenacapavir or lenacapavir sodium) at a concentration of about 309 mg/mL.

The solution provided herein may comprise lenacapavir, or a pharmaceutically acceptable salt thereof, PEG 300, and water. The solution may comprise lenacapavir sodium, PEG 300, and water. The solution may comprise lenacapavir, PEG 300, and water.

The amount of water in the solution comprising lenacapavir sodium, PEG 300, and water may be about 10 w/w % to about 40 w/w %. The amount of water in the solution comprising lenacapavir sodium, PEG 300, and water may be about 15 w/w % to about 35 w/w %. The amount of water in the solution comprising lenacapavir sodium, PEG 300, and water may be about 20 w/w % to about 30 w/w %. The amount of water in the solution comprising lenacapavir sodium, PEG 300, and water may be about 21 w/w % to about 29 w/w %.

The amount of water in the solution comprising lenacapavir sodium, PEG 300, and water may be about 21.1 w/w % to about 27.5 w/w %. The amount of water in the solution comprising lenacapavir sodium, PEG 300, and water may be about 21.13 w/w % to about 27.47 w/w %. The amount of water in the solution comprising lenacapavir sodium, PEG 300, and water may be about 21.1 w/w %. The amount of water in the solution comprising lenacapavir sodium, PEG 300, and water may be about 21.13 w/w %. The amount of water in the solution comprising lenacapavir sodium, PEG 300, and water may be about 23 w/w %. The amount of water in the solution comprising lenacapavir sodium, PEG 300, and water may be about 23.4 w/w %. The amount of water in the solution comprising lenacapavir sodium, PEG 300, and water may be about 23.41 w/w %.

The amount of PEG 300 in the solution comprising lenacapavir sodium, PEG 300, and water may be about 35 w/w % to about 75 w/w %. The amount of PEG 300 in the solution comprising lenacapavir sodium, PEG 300, and water may be about 40 w/w % to about 55 w/w %. The amount of PEG 300 in the solution comprising lenacapavir sodium, PEG 300, and water may be about 43 w/w % to about 47 w/w %. The amount of PEG 300 in the solution comprising lenacapavir sodium, PEG 300, and water may be about 45 w/w %. The amount of PEG 300 in the solution comprising lenacapavir sodium, PEG 300, and water may be about 45.25 w/w %. The amount of PEG 300 in the solution comprising lenacapavir sodium, PEG 300, and water may be about 48 w/w % to about 52 w/w %. The amount of PEG 300 in the solution comprising lenacapavir sodium, PEG 300, and water may be about 50 w/w %. The amount of PEG 300 in the solution comprising lenacapavir sodium, PEG 300, and water may be about 50.1 w/w %. The amount of PEG 300 in the solution comprising lenacapavir sodium, PEG 300, and water may be about 50.13 w/w %.

The amount of lenacapavir sodium in the solution comprising PEG 300 and water may be about 5 w/w % to about 35 w/w %. The amount of lenacapavir sodium in the solution comprising PEG 300 and water may be about 20 w/w % to about 35 w/w %.

The amount of lenacapavir sodium in the solution comprising PEG 300 and water may be about 20 w/w % to about 30 w/w %. The amount of lenacapavir sodium in the solution comprising PEG 300 and water may be about 24 w/w % to about 28 w/w %. The amount of lenacapavir sodium in the solution comprising PEG 300 and water may be about 26.5 w/w %. The amount of lenacapavir sodium in the solution comprising PEG 300 and water may be about 26.46 w/w %.

The amount of lenacapavir sodium in the solution comprising PEG 300 and water may be about 30 w/w % to about 35 w/w %. The amount of lenacapavir sodium in the solution comprising PEG 300 and water may be about 32 w/w % to about 34 w/w %. The amount of lenacapavir sodium in the solution comprising PEG 300 and water may be about 33.6 w/w %. The amount of lenacapavir sodium in the solution comprising PEG 300 and water may be about 33.61 w/w %.

The solution may comprise about 10 w/w % to about 40 w/w % water, about 35 w/w % to about 75 w/w % PEG 300, and about 5 w/w % to about 45 w/w % of lenacapavir sodium. The solution may comprise about 10 w/w % to about 30 w/w % water, about 35 w/w % to about 65 w/w % PEG 300, and about 5 w/w % to about 45 w/w % of lenacapavir sodium.

The solution may comprise about 21.13 w/w % to about 27.47 w/w % water, about 45.25 w/w % to about 58.84 w/w % PEG 300, and about 13.69 w/w % to about 33.61 w/w % of lenacapavir sodium. The solution may comprise about 21.1 w/w % water, about 45.3 w/w % PEG 300, and about 33.6 w/w % of lenacapavir sodium. The solution may comprise about 21.13 w/w % water, about 45.25 w/w % PEG 300, and about 33.61 w/w % of lenacapavir sodium. The solution may comprise about 23.4 w/w % water, about 50.1 w/w % PEG 300, and about 26.5 w/w % of lenacapavir sodium. The solution may comprise about 23.41 w/w % water, about 50.13 w/w % PEG 300, and about 26.46 w/w % of lenacapavir sodium.

The solution provided herein may further comprise ethanol. The solution may comprise about 10 w/w % to about 40 w/w % water, about 20 w/w % to about 75 w/w % PEG 300, about 10 w/w % to about 70 w/w % of lenacapavir sodium, and about 1 w/w % to about 10 w/w % of ethanol. The solution may comprise about 10 w/w % to about 20 w/w % water, about 30 w/w % to about 40 w/w % PEG 300, about 37 w/w % to about 45 w/w % of lenacapavir sodium, and about 3 w/w % to about 8 w/w % of ethanol.

The solution may comprise about 16.93 w/w % water, about 36.22 w/w % PEG 300, about 41.85 w/w % of lenacapavir sodium, and about 5.00 w/w % ethanol. The solution may comprise about 16.9 w/w % water, about 36.2 w/w % PEG 300, about 41.9 w/w % of lenacapavir sodium, and about 5.0 w/w % ethanol.

The medicament which is administered by the injection device of the invention may be a long-acting injectable formulation which can be administered to a patient, for example, twice per month, once per month, once per quarter (e.g., every 3 months), twice per year (e.g., every 6 months), once per year, or less frequently.

Additionally, the medicament can include a formulation which has relatively high viscosity. The medicament may have a viscosity of 100-1000 cP. Preferably, the medicament may have a viscosity of 100-600 cP. For example, the medicament may have a viscosity of 250-500 cP, 150-350 cP or 450-600 cP. The medicament may be a solution comprising lenacapavir sodium and the medicament may have a viscosity of 250-500 cP, 150-350 cP or 450-600 cP.

The sensing assembly (e.g., the main PCB of autoinjectors 100, 200, 500, 700, 800, or 1000) is intended to include various forms of digital computers, such as printed circuit boards (PCB), processors, digital circuitry, or otherwise parts of a system for determining dosing progression of an autoinjector. Additionally, the system can include portable storage media, such as, Universal Serial Bus (USB) flash drives. For example, the USB flash drives may store operating systems and other applications. The USB flash drives can include input/output components, such as a wireless transmitter or USB connector that may be inserted into a USB port of another computing device.

The sensing assembly can include a processor, a memory, a storage device, and an input/output device (for example, sensors). Each of the components is interconnected using a system bus. The sensing assembly is capable of processing instructions for execution within the sensing assembly. The sensing assembly may be designed using any of a number of architectures. For example, the sensing assembly can include a CISC (Complex Instruction Set Computers) processor, a RISC (Reduced Instruction Set Computer) processor, or a MISC (Minimal Instruction Set Computer) processor.

In one implementation, the processor is a single-threaded processor. In another implementation, the processor is a multi-threaded processor. The processor is capable of processing instructions stored in the memory or on the storage device to display graphical information for a user interface on an input/output device.

The memory stores information within the sensing assembly. In one implementation, the memory is a computer-readable medium. In one implementation, the memory is a volatile memory unit. In another implementation, the memory is a non-volatile memory unit.

The storage device is capable of providing mass storage for the sensing assembly. In one implementation, the storage device is a computer-readable medium. In various different implementations, the storage device may be a floppy disk device, a hard disk device, an optical disk device, or a tape device.

The features described can be implemented in digital electronic circuitry, or in computer hardware, firmware, software, or in combinations of them. The features can be implemented in a computer program product tangibly embodied in an information carrier, for example, in a machine-readable storage device for execution by a programmable processor; and method steps can be performed by a programmable processor executing a program of instructions to perform functions of the described implementations by operating on input data and generating output. The described features can be implemented advantageously in one or more computer programs that are executable on a programmable system including at least one programmable processor coupled to receive data and instructions from, and to transmit data and instructions to, a data storage system, at least one input device, and at least one output device. A computer program is a set of instructions that can be used, directly or indirectly, in a computer to perform a certain activity or bring about a certain result. A computer program can be written in any form of programming language, including compiled or interpreted languages, and it can be deployed in any form, including as a stand-alone program or as a module, component, subroutine, or other unit suitable for use in a computing environment.

Suitable processors for the execution of a program of instructions include, by way of example, both general and special purpose microprocessors, and the sole processor or one of multiple processors of any kind of computer. Generally, a processor will receive instructions and data from a read-only memory or a random access memory or both. The essential elements of a computer are a processor for executing instructions and one or more memories for storing instructions and data. Generally, a computer will also include, or be operatively coupled to communicate with, one or more mass storage devices for storing data files; such devices include magnetic disks, such as internal hard disks and removable disks; magneto-optical disks; and optical disks. Storage devices suitable for tangibly embodying computer program instructions and data include all forms of non-volatile memory, including by way of example semiconductor memory devices, such as EPROM (erasable programmable read-only memory), EEPROM (electrically erasable programmable read-only memory), and flash memory devices; magnetic disks such as internal hard disks and removable disks; magneto-optical disks; and CD-ROM and DVD-ROM disks. The processor and the memory can be supplemented by, or incorporated in, ASICs (application-specific integrated circuits).

The features can be implemented in a control system that includes a back-end component, such as a data server, or that includes a middleware component, such as an application server or an Internet server, or that includes a front-end component, such as a client computer having a graphical user interface or an Internet browser, or any combination of them. The components of the system can be connected by any form or medium of digital data communication such as a communication network. Examples of communication networks include a local area network (“LAN”), a wide area network (“WAN”), peer-to-peer networks (having ad-hoc or static members), grid computing infrastructures, and the Internet.

A number of embodiments have been described. Nevertheless, it will be understood that various modifications may be made without departing from the spirit and scope of the disclosure. Accordingly, other embodiments are within the scope of the following claims.

### Embodiments

The following list provides embodiments of the invention and forms part of the description. These embodiments can be combined in any compatible combination beyond those expressly stated. The embodiments can also be combined with any compatible features described herein:

1. An autoinjector comprising:
a housing;
a needle arranged at a distal end of the housing;
a container disposed within the housing and configured to contain medicament;
a plunger slidably disposed within the container;
a plunger rod configured to push the plunger through the container to dispense the medicament through the needle when the container contains medicament, wherein the plunger rod comprises one or more conductive regions;
a first conductive contact configured to contact a portion of the plunger rod, wherein a dispensing movement of the plunger rod generates a first electrical signal upon contacting said first conductive contact and at least one of said one or more conductive regions of the plunger rod; and
a first sensor configured to detect said first electrical signal as the plunger rod moves.
   2. The autoinjector of embodiment 1, wherein the first electrical signal is indicative of dose progression.
   3. The autoinjector of embodiment 1 or embodiment 2, wherein the container contains the medicament.
   4. The autoinjector of any preceding embodiment, wherein the medicament comprises lenacapavir or a pharmaceutically accepted salt thereof.
   5. The autoinjector of embodiment 4, wherein the medicament comprises lenacapavir sodium.
   6. The autoinjector of embodiment 5, wherein the medicament is a solution comprising: lenacapavir sodium, PEG 300 and water.
   7. The autoinjector of embodiment 6, wherein the solution comprises about 21.13 w/w % water, about 45.25 w/w % PEG 300, and about 33.61 w/w % of lenacapavir sodium.
   8. The autoinjector of embodiment 6, wherein the solution comprises about 23.41 w/w % water, about 50.13 w/w % PEG 300, and about 26.46 w/w % of lenacapavir sodium.
   9. The autoinjector of embodiment 6, wherein the solution further comprises ethanol, optionally wherein the solution comprises about 16.9 w/w % water, about 36.2 w/w % PEG 300, about 41.9 w/w % of lenacapavir sodium, and about 5.0 w/w % ethanol.
   10. The autoinjector of any preceding embodiment, wherein the autoinjector further comprises a gas canister assembly configured to release pressurized gas which, when released, provides a force acting on the plunger rod to push the plunger through the container.
   11. The autoinjector of any preceding embodiment, wherein the first conductive contact comprises a lock ring contact configured to be attached to a lock ring.
   12. The autoinjector of embodiment 11, wherein the lock ring contact and/or the lock ring comprises an opening configured to provide a pathway for the dispensing movement of the plunger rod.
   13. The autoinjector of embodiment 12, wherein the lock ring contact comprises one or more deflectable contacts disposed around said opening and configured to contact at least one of said one or more conductive regions during the dispensing movement of the plunger rod.
   14. The autoinjector of any one of embodiments 1-13, wherein the plunger rod is configured to generate a plurality of electrical signals during the dispensing movement of the plunger rod.
   15. The autoinjector of embodiment 14, wherein the plunger rod comprises a plurality of conductive regions.
   16. The autoinjector of any one of embodiments 1-15, wherein the first electrical signal is configured to indicate completion of the dispensing movement of the plunger rod.
   17. The autoinjector of embodiment 15, wherein a first conductive region of the plurality of conductive regions is configured to indicate dose start of the medicament, and/or wherein a last conductive region of the plurality of conductive regions is configured to indicate dose end of the medicament.
   18. The autoinjector of embodiments 10 or 17, wherein each conductive region of the plurality of conductive regions is configured to generate a corresponding electrical signal during the dispensing movement of the plunger rod.
   19. The autoinjector of any one of embodiments 1-18, wherein the first conductive contact is configured to generate a plurality of electrical signals during the dispensing movement of the plunger rod.
   20. The autoinjector of embodiment 19, wherein the first conductive contact comprises an opening and one or more deflectable contacts disposed around said opening.
   21. The autoinjector of embodiment 20, wherein the one or more deflectable contacts are configured to contact each conductive region during the dispensing movement of the plunger rod.
   22. The autoinjector of any one of embodiments 1-21, further comprising:
an audible clicker configured to produce one or more audible clicks during the dispensing movement of the plunger rod.
   23. The autoinjector of embodiment 22, wherein the audible clicker comprises a ring surrounding the plunger rod, and wherein the ring comprises a deflectable protrusion configured to contact the plunger rod and produce the one or more audible clicks.
   24 The autoinjector of embodiment 23, wherein the plunger rod comprises a ridged surface configured to contact the deflectable protrusion of the audible clicker.
   25. The autoinjector of embodiment 24, wherein each ridge of the ridged surface is configured to cause the deflectable protrusion to deflect during the dispensing movement of the plunger rod.
   26. The autoinjector of any one of embodiments 1-25, wherein the first sensor is disposed between the proximal end and a distal end of the housing.
   27. The autoinjector of any one of embodiments 1-26, wherein the first sensor comprises a voltage sensor or a resistance sensor.
   28. The autoinjector of any one of embodiments 1-27, wherein the first sensor is disposed on a surface of a printed circuit board.
   29 The autoinjector of any one of embodiments 1-28, wherein the first sensor is further configured to detect a second electrical signal generated by applying an insertion force to insert the needle into a user.
   30. The autoinjector of any one of embodiments 1-28, wherein a second sensor is disposed between a proximal end and a distal end of the housing, and wherein the second sensor is configured to detect a second electrical signal generated by applying an insertion force to insert the needle into a user.
   31. The autoinjector of embodiment 30, wherein the second sensor comprises a voltage sensor or a resistance sensor.
   32. The autoinjector of any one of embodiments 1-29, wherein a third sensor is disposed between a proximal end and a distal end of the housing, and wherein the third sensor is configured to detect an ambient temperature in proximity to the container.
   33. The autoinjector of embodiment 32, wherein the third sensor comprises a thermistor.
   34. An autoinjector comprising:
a housing;
a needle arranged at a distal end of the housing;
a container disposed within the housing and configured to contain medicament;
a plunger slidably disposed within the container;
a plunger rod configured to push the plunger through the container to dispense the medicament through the needle when the container contains medicament, wherein the plunger rod comprises one or more conductive regions;
a mechanism configured so that a dispensing movement of the plunger rod causes the mechanism to generate a first electrical signal from at least one of said one or more conductive regions; and
a first sensor configured to detect said first electrical signal generated by the mechanism.
   35. An autoinjector comprising:
a housing;
a needle arranged at a distal end of the housing;
a needle guard configured to expose the needle when an insertion force is applied to insert the needle into a user, wherein the needle guard comprises a needle guard contact;
a container disposed within the housing and configured to contain medicament;
a plunger slidably disposed within the container;
a plunger rod configured to push the plunger through the container to dispense the medicament through the needle when the container contains medicament and when the needle is exposed, wherein the plunger rod comprises one or more conductive regions;
a lock ring contact configured to contact a portion of the plunger rod, wherein a dispensing movement of the plunger rod generates a first electrical signal upon contacting said lock ring contact and at least one of said one or more conductive regions of the plunger rod, and wherein a compression movement of the needle guard generates a second electrical signal upon contacting said lock ring contact and said needle guard contact; and
a first sensor configured to detect said first electrical signal as the plunger rod moves and/or said second electrical signal as the needle guard moves.
   36. A system comprising:
an autoinjector of any one of embodiments 1-35; and
a processor configured to process data generated by the sensor to track the dispensing movement of the plunger rod.
   37. A method of detecting a dispensing movement of a plunger rod within an autoinjector, the method comprising:
detecting, using a first sensor, one or more electrical signals of a first conductive contact as the plunger rod moves during the dispensing movement, wherein the plunger rod comprises one or more conductive regions, and
wherein the dispensing movement of the plunger rod provides electrical communication between one or more conductive regions and the first conductive contact causing the first conductive contact to generate the one or more electrical signals.
   38. A method of detecting a dispensing movement of a plunger rod within an autoinjector, the method comprising:
detecting an electrical signal generated due to movement of the plunger rod during the dispensing movement.
   39. The method of embodiment 37 or 38, wherein the plunger rod is configured to generate a plurality of electrical signals during the dispensing movement of the plunger rod.
   40. The method of embodiment 39, wherein the plunger rod comprises a plurality of conductive regions.
   41. The method of embodiment 40, wherein a first conductive region of the plurality of conductive regions is configured to indicate dose start of the medicament, and/or wherein a last conductive region of the plurality of conductive regions is configured to indicate dose end of the medicament.
   42. The method of embodiment 40 or 41, wherein each conductive region of the plurality of conductive regions is configured to generate a corresponding electrical signal during the dispensing movement of the plunger rod.
   43. The method of any one of embodiments 37-42, wherein a lock ring contact is configured to generate a plurality of electrical signals during the dispensing movement of the plunger rod.
   44. The method of embodiment 43, wherein the lock ring contact comprises an opening and one or more deflectable contacts disposed around said opening.
   45. The method of embodiment 44, wherein the one or more deflectable contacts are configured to contact each conductive region during the dispensing movement of the plunger rod.
   46. The method of any one of embodiments 37-45, wherein the autoinjector further comprises an audible clicker configured to produce one or more audible clicks during the dispensing movement of the plunger rod.
   47. The method of embodiment 46, wherein the audible clicker comprises a ring surrounding the plunger rod, and wherein the ring comprises a deflectable protrusion configured to contact the plunger rod and produce the one or more audible clicks.
   48. The method of embodiment 47, wherein the plunger rod comprises a ridged surface configured to contact the deflectable protrusion of the audible clicker.
   49. The method of embodiment 48, wherein each ridge of the ridged surface is configured to cause the deflectable protrusion to deflect during the dispensing movement of the plunger rod.
   50. The method of embodiment 37, wherein the first sensor is disposed between a proximal end and a distal end of the housing.
   51. The method of embodiment 37 or 45, wherein the first sensor comprises a voltage sensor.
   52. The method of any of embodiments 37, 45 or 46, wherein the first sensor is disposed on a surface of a printed circuit board.
   53. The method of any of embodiments 37, 45, 46 or 47, wherein the first sensor is further configured to detect a second electrical signal generated by applying an insertion force to insert the needle into a user.
   54. The method of any of embodiments 37-52, wherein a second sensor is disposed between a proximal end and a distal end of the housing, and wherein the second sensor is configured to detect a second electrical signal generated by applying an insertion force to insert the needle into a user.
   55. The method of embodiment 54, wherein the second sensor comprises a voltage sensor or a resistance sensor.
   56. The method of any of embodiments 37-55, wherein a third sensor is disposed between a proximal end and a distal end of the housing, and wherein the third sensor is configured to detect an ambient temperature in proximity to the container.
   57. The method of embodiment 56, wherein the third sensor comprises a thermistor.
   58. The method of any one of embodiments 37-57, further comprising sending, via a wireless transfer protocol module, signals from a first sensor, if present, and a second sensor, if present, to a mobile device.
   59. The method of embodiment 58, further comprising displaying information about the dispensing movement on the mobile device.
   60. The method of any one of embodiments 37-59, further comprising measuring a temperature of medicament within the autoinjector or an ambient temperature within the autoinjector using a temperature sensor.
   61. The method of embodiment 60, further comprising sending, via a wireless transfer protocol module, signals from the temperature sensor to a mobile device.
   62. The method of embodiment 61, further comprising displaying the temperature of the medicament or the ambient temperature on the mobile device; indicating that the temperature of the medicament or the ambient temperature is above a threshold temperature for use of the autoinjector; or both.
   63. A composition comprising lenacapavir or a pharmaceutically accepted salt thereof for use in the prevention or treatment of HIV, wherein the composition is administered via the autoinjector of any of embodiments 1 to 36.
   64. The composition of embodiment 63, wherein the composition is subcutaneously administered via the autoinjector, optionally wherein the medicament is a solution comprising either:
(a) about 21.13 w/w % water, about 45.25 w/w % PEG 300, and about 33.61 w/w % of lenacapavir sodium; or
(b) about 23.41 w/w % water, about 50.13 w/w % PEG 300, and about 26.46 w/w % of lenacapavir sodium.
   65. The composition of embodiment 63, wherein the composition is intramuscularly administered via the autoinjector, optionally wherein the composition is a solution comprising about 16.9 w/w % water, about 36.2 w/w % PEG 300, about 41.9 w/w % of lenacapavir sodium, and about 5.0 w/w % ethanol.
   66. Use of lenacapavir or a pharmaceutically accepted salt thereof for the manufacture of a medicament for the prevention or treatment of HIV, wherein the prevention or treatment comprises administering the medicament subcutaneously via the autoinjector of any of embodiments 1 to 36.
   67. The use of embodiment 66, wherein the medicament is subcutaneously administered via the autoinjector, optionally wherein the medicament is a solution comprising either:
(a) about 21.13 w/w % water, about 45.25 w/w % PEG 300, and about 33.61 w/w % of lenacapavir sodium; or
(b) about 23.41 w/w % water, about 50.13 w/w % PEG 300, and about 26.46 w/w % of lenacapavir sodium.
   68. The use of embodiment 66, wherein the medicament is intramuscularly administered via the autoinjector, optionally wherein the medicament is a solution comprising about 16.9 w/w % water, about 36.2 w/w % PEG 300, about 41.9 w/w % of lenacapavir sodium, and about 5.0 w/w % ethanol.

1. An autoinjector comprising:
a housing;
a needle arranged at a distal end of the housing;
a container disposed within the housing and configured to contain medicament;
a plunger slidably disposed within the container;
a plunger rod configured to push the plunger through the container to dispense the medicament through the needle when the container contains medicament, wherein the plunger rod comprises one or more conductive regions;
a first conductive contact configured to contact a portion of the plunger rod, wherein a dispensing movement of the plunger rod generates a first electrical signal upon contacting said first conductive contact and at least one of said one or more conductive regions of the plunger rod; and
a first sensor configured to detect said first electrical signal as the plunger rod moves.

2. The autoinjector of claim 1, wherein the first electrical signal is indicative of dose progression.

3. (canceled)

4. The autoinjector of claim 1, wherein the medicament comprises lenacapavir or a pharmaceutically accepted salt thereof.

5. The autoinjector of claim 1, wherein the autoinjector further comprises a gas canister assembly configured to release pressurized gas which, when released, provides a force acting on the plunger rod to push the plunger through the container.

6. The autoinjector of claim 1, wherein the first conductive contact comprises a lock ring contact configured to be attached to a lock ring.

7. The autoinjector of claim 6, wherein the lock ring contact and/or the lock ring comprises an opening configured to provide a pathway for the dispensing movement of the plunger rod, and wherein the lock ring contact comprises one or more deflectable contacts disposed around said opening and configured to contact at least one of said one or more conductive regions during the dispensing movement of the plunger rod.

8. (canceled)

9. The autoinjector of claim 0, wherein the plunger rod comprises a plurality of conductive regions and is configured to generate a plurality of electrical signals during the dispensing movement of the plunger rod.

10-11. (canceled)

12. The autoinjector of claim 9, wherein a first conductive region of the plurality of conductive regions is configured to indicate dose start of the medicament, and/or wherein a last conductive region of the plurality of conductive regions is configured to indicate dose end of the medicament.

13-16. (canceled)

17. The autoinjector of claim 0, further comprising:
an audible clicker configured to produce one or more audible clicks during the dispensing movement of the plunger rod.

18. The autoinjector of claim 17, wherein the audible clicker comprises a ring surrounding the plunger rod, and wherein the ring comprises a deflectable protrusion configured to contact the plunger rod and produce the one or more audible clicks.

19. The autoinjector of claim 18, wherein the plunger rod comprises a ridged surface configured to cause the deflectable protrusion of the audible clicker to deflect during the dispensing movement of the plunger rod.

20-21. (canceled)

22. The autoinjector of claim 1, wherein the first sensor comprises a voltage sensor or a resistance sensor.

23. The autoinjector of claim 1, wherein the first sensor is disposed on a surface of a printed circuit board disposed between a proximal end and a distal end of the housing.

24. The autoinjector of claim 1, wherein the first sensor is further configured to detect a second electrical signal generated by applying an insertion force to insert the needle into a user.

25-26. (canceled)

27. The autoinjector of claim 1, wherein a second sensor is disposed between a proximal end and a distal end of the housing, and wherein the second sensor is configured to detect an ambient temperature in proximity to the container.

28-31. (canceled)

32. A method of detecting a dispensing movement of a plunger rod within an autoinjector, the method comprising:
detecting, using a first sensor, one or more electrical signals of a first conductive contact as the plunger rod moves during the dispensing movement, wherein the plunger rod comprises one or more conductive regions, and
wherein the dispensing movement of the plunger rod provides electrical communication between one or more conductive regions and the first conductive contact causing the first conductive contact to generate the one or more electrical signals.

33. (canceled)

34. The method of claim 32, wherein the plunger rod comprises a plurality of conductive regions and to the method further comprises generating a plurality of electrical signals during the dispensing movement of the plunger rod.

35. (canceled)

36. The method of claim 34, wherein a first conductive region of the plurality of conductive regions is configured to indicate dose start of a medicament, and/or wherein a last conductive region of the plurality of conductive regions is configured to indicate dose end of the medicament.

37. (canceled)

38. The method of claim 32, wherein a lock ring contact is configured to generate a plurality of electrical signals during the dispensing movement of the plunger rod.

39. The method of claim 38, wherein the lock ring contact comprises an opening and one or more deflectable contacts disposed around said opening, and wherein during the dispensing movement of the plunger rod, the one or more deflectable contacts contact each conductive region.

40. (canceled)

41. The method of claim 32, wherein the autoinjector further comprises an audible clicker configured to produce one or more audible clicks during the dispensing movement of the plunger rod.

42. The method of claim 41, wherein the audible clicker comprises a ring surrounding the plunger rod, and wherein the ring comprises a deflectable protrusion configured to contact the plunger rod and produce the one or more audible clicks.

43. The method of claim 42, wherein the plunger rod comprises a ridged surface configured to contact the deflectable protrusion of the audible clicker, and wherein during the dispensing movement of the plunger rod, ridges of the ridged surface cause the deflectable protrusion to deflect.

44-47. (canceled)

48. The method of claim 32, further comprising detecting, by the first sensor, a second electrical signal generated by applying an insertion force to insert a needle into a user.

49. The method of claim 32, wherein a second sensor is disposed between a proximal end and a distal end of the housing, and wherein the method further comprises detecting, by the second sensor, a second electrical signal generated by applying an insertion force to insert a needle into a user.

50-52. (canceled)

53. The method of claim 32, further comprising sending, via a wireless transfer protocol module, signals from a first sensor, if present, and a second sensor, if present, to a mobile device.

54. The method of claim 53, further comprising displaying information about the dispensing movement on the mobile device.

55. The method of claim 32, further comprising measuring a temperature of medicament within the autoinjector or an ambient temperature within the autoinjector using a temperature sensor.

56. (canceled)

57. The method of claim 55, further comprising displaying the temperature of the medicament or the ambient temperature on a mobile device; indicating that the temperature of the medicament or the ambient temperature is above a threshold temperature for use of the autoinjector; or both.

58. A composition comprising lenacapavir or a pharmaceutically accepted salt thereof for use in the prevention or treatment of HIV, wherein the composition is administered via the autoinjector of claim 1.

59. Use of lenacapavir or a pharmaceutically accepted salt thereof for the manufacture of a medicament for the prevention or treatment of HIV, wherein the prevention or treatment comprises administering the medicament via the autoinjector of claim 1.
